(19) United States
(12) Patent Application Publication
(10) Pub. No.: US 2022/0372455 A1
(43) Pub. Date: Nov. 24, 2022

(54) CRISPR TYPE V-U1 SYSTEM FROM MYCOBACTERIUM MUCOGENICUM AND USES THEREOF

(71) Applicant: Wageningen Universiteit, Wagenin (NL)

(72) Inventors: John Van Der Oost, Renkum (NL); Prarthana Mohanraju, Leiden (NL); Wen Ying Wu, Wagening (NL); Sjoerd Creutzburg, Bennekom (NL)

(21) Appl. No.: 17/624,439

(22) PCT Filed: Jul. 3, 2020
(86) PCT No.: PCT/EP2020/068824

§ 371 Date: Jan. 3, 2022

(30) Foreign Application Priority Data
Jul. 3, 2019 (GB) 1909597.5

(51) Int. Cl. C12N 9/22; C12N 15/11; C12N 9/78; C12N 15/62; C12N 15/64; C12N 15/90; C07K 16/46

(52) U.S. Cl. CPC C12N 9/22; C12N 15/11; C12Y 305/04005; C12N 9/78; C12N 15/625; C12N 15/64; C12N 15/90; C07K 16/46; C12N 2800/80; C12N 2310/20; C12N 2800/10; C07K 2319/00

(57) ABSTRACT

The type V-U1 system from the bacterium Mycobacterium mucogenicum CCH10-A2 (Mmu) has a nuclease which binds dsDNA but it does not cleave it. Additionally, after dsDNA binding by the nuclease an RuvC-dependent interference of nascent transcript (mRNA) takes place and this mechanism has not been described before for any CRISPR system. CRISPR based gene manipulation can therefore use CRISPR endonucleases from the Type V-U1 system from Mycobacterium mucogenicum, including variant and modified endonucleases, so as to provide for methods of expression control and gene editing in cells of any living organism or of any nucleic acid in vitro.

This invention relates to CRISPR based gene manipulation and to CRISPR endonucleases from the Type V-U1 system from Mycobacterium mucogenicum, including variant and modified endonucleases, so as to provide for methods of expression control and gene editing in cells of any living organism or of any nucleic acid in vitro.

### BACKGROUND

CRISPR-Cas, originally an immune system found in bacteria, has been repurposed as a genome editing tool to modify DNA of living organisms. This genome editing tool utilizes DNA endonucleases such as Cas9 and Cas12a (Cpf1). A major drawback for both proteins are their size, as they are slightly too big for AAV delivery (adeno-associated virus) into mammalian cells.

Shmakov S., Smargon A., Scott D., et al. (2017) “Diversity and evolution of class 2 CRISPR-Cas systems” Nat Rev Microbiol 15: 169-182 describes a new computational pipeline for use in discovering new Class 2 CRISPR-Cas systems. Clustering and phylogenetic analysis was done both based on the presence of a Cas1 homolog and/or the presence of a CRISPR array. A non-redundant, representative sequence set was constructed using the NCBI BLASTCLUST program 2. (ftp://ftp.ncbi.nih.gov/blast/documents/blastclust.html) with sequence identity threshold of 90% and length coverage threshold of 0.9. The longest sequence was selected as the reference sequence. Permissive clustering of sequences was performed using UCLUST 3, with sequence similarity threshold of 0.3. Multiple alignments of protein sequences were constructed using MUSCLE 4 and MAFFT 5 programs. New systems were found. These new systems are tentatively classified as type V-U1, V-U2, V-U3, V-U4 and V-U5. However, the study is purely based on sequence information and there is no functionality tested for or described for any of the subtype V-U effectors. As stated by Shmakov et al., because there is no bona tide CRISPR response for the subtype V-U effectors no naming is undertaken.

A recent, more complete and more accurate tree of the Type V nucleases U 1-5 is provided by Yan W X et al (2019) “Functionally diverse type V CRISPR-Cas systems” Science 363: 88-91. doi: 10.1126/science.aav7271. Epub 2018 Dec. 6.
Ayman, E. et al., (2018) “CRISPR base editors: genome editing without double-stranded breaks” Biochemical Journal 475: 1955-1964. This is a review article which summarises state of knowledge about DNA base editors which are constructed from a Cas9 or Cpf1 backbone.
Engineered versions of Cas9 and Cas12a are known which can be used as base editors of DNA. Komor, Alexis C., et al. (2016) “Programmable editing of a target base in genomic DNA without double-stranded DNA cleavage.” Nature 533: 420-424 describes a Cas9 base editor.
Ma, Yunqing, et al. (2016) “Targeted AID-mediated mutagenesis (TAM) enables efficient genomic diversification in mammalian cells.” Nature Methods 13: 1029-1035 describes a Cas9 BE2 (Target AID) base editor.
Li, Xiaosa, et al. (2018) “Base editing with a Cpf1-cytidine deaminase fusion.” Nature Biotechnology 36: 324-327 describes a Cpf1 base editor.
Banno, Satomi, et al. (2018) “Deaminase-mediated multiplex genome editing in Escherichia coli” Nature Microbiology 3: 423-429 describes a Cas9 base editor for use in bacteria base editing. A cytidine deaminase (Petromyzon marinus PmCDA1) is fused to a catalytically inactive Cas9 and is under the control of a temperature-inducible promoter, and a 20 nt target sequence single guide RNA (sgRNA) under the control of a synthetic constitutive promoter.
Harrington, Lucas B., et al. (2018) “Programmed DNA destruction by miniature CRISPR-Cas14 enzymes.” Science 362: 839-842 describes a set of CRISPR-Cas systems from uncultivated archaea that contain Cas14, a family of exceptionally compact RNA-guided nucleases (400 to 700 amino acids). Despite their small size, Cas14 proteins are capable of targeted single-stranded DNA (ssDNA) cleavage without restrictive sequence requirements. Moreover, target recognition by Cas14 triggers nonspecific cutting of ssDNA molecules, an activity that enables high-fidelity single-nucleotide polymorphism genotyping (Cas14-DETECTR). Metagenomic data show that multiple CRISPR-Cas14 systems evolved independently and suggest a potential evolutionary origin of single-effector CRISPR-based adaptive immunity.
WO 2018/035388 A1 (BROAD INST INC) discloses engineered CRISPR-Cas effector proteins comprising a Type V effector protein such as Cpf1. Many modifications and deactivated Cpf1 are described. Also a viral vector for delivery of such CRISPR-Cas effector proteins. Compact promoters are referred to for packing and thus expressing larger transgenes for targeted delivery and tissue-specificity.
WO2018/191715 A2 (SYNTHETIC GENOMICS) describes Mmc3 polypeptides having nuclease activity. The Mmc3 polypeptides function as Class 2 Type V effectors, and catalyze double stranded breaks in nucleic acid strands. The polypeptides are useful, for example, for gene editing systems such as CRISPR, to make site specific alterations of target nucleic acid sequences.

### BRIEF SUMMARY OF THE DISCLOSURE

The inventors have surprisingly discovered that the type V-U1 system from the bacterium Mycobacterium mucogenicum CCH10-A2 (Mmu) is particularly advantageous in many respects.

Indeed, a major surprise of the V-U1 system (Mmu nuclease) is that it binds dsDNA but it does not cleave it; in addition, a second major surprise, is that after dsDNA binding (that may trigger RuvC activation) an RuvC-dependent interference (probably degradation) of nascent transcript (mRNA) is observed: such a mechanism has not been described before for any CRISPR system.

In accordance with the present invention there is provided a polynucleotide comprising a MmuC2c4 endonuclease encoded by a nucleotide sequence as set forth in SEQ ID NO:2 or a sequence of at least 55% identity thereto; or SEQ ID NO: 3 or a sequence of at least 55% identity thereto.

The MmuC2c4 of the invention may bind to any form of dsDNA at a target locus as directed by the guiding RNA with which it associates, without cleaving it. That is to say the DNA may be a dsDNA and preferably such dsDNA may be comprised in native genomic DNA, e.g. chromosomal DNA, whether extracted from a nucleus in vitro, or in the form of nuclear material or a nucleus, including in a cell-free system, or in vitro in an isolated cell or cell culture or tissue, or in vivo in an organism, whether prokaryote or eukaryote.

Without wishing to be bound by any particular theory, the inventors believe that after guide-dependent DNA targeting (binding) by the MmuC2c4 or dMmuC2c4 of the invention, gene silencing can occur through blocking of transcription (roadblock for RNA polymerase) and in addition, in case of an active Mmu, through targeting of the nascent RNA. All forms of transcribed RNA may be targeted by the ribonucleoprotein complexes of the invention comprising the described Mmu CRISPR type V-U1 nuclease.

The RNA which may be degraded or digested by the MmuC2c4 of the invention can be selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), small cytoplasmic RNA (scRNA) and CRISPR RNA (crRNA).

The modifying of RNA by using a MmuC2c4 of the invention has a wide variety of utility including inactivating a target RNA of any kind of cell from any kind of organism, including prokaryote or eukaryote. The invention therefore has a broad spectrum of applications for example in gene therapy, drug screening, disease diagnosis, and prognosis.

The invention also provides an expression vector comprising a mmuc2c4 RNA endonuclease encoded by a nucleotide sequence as set forth in SEQ ID NO:2 or a sequence of at least 55% identity thereto, under the control of a suitable expression promoter. The expression vector may further comprise a nucleotide sequence comprising an expression promoter and a sequence under the control of the promoter encoding a guide RNA (gRNA) with a nucleotide sequence which recognises a target locus of interest and the PAM sequence of 5′ TTM, 5′ NTTM or 5′ CTM in dsDNA.

The invention further includes a cell comprising an expression vector as hereinbefore defined.

A cell of the invention may further comprise a second expression vector which comprises an expression promoter and a sequence under the control of the promoter which encodes a guide RNA (gRNA) having a nucleotide sequence which recognises a target locus of interest and the PAM sequence of 5′ TTM, 5′ NTTM or 5′ CTM in dsDNA.

The invention also include a method of repressing or interfering with the expression of a target gene sequence by an organism or cell thereof, or in a cell-free expression system, comprising exposing double stranded DNA (dsDNA) comprising the target gene sequence to an MmuC2c4 RNA endonuclease, and a guide RNA which directs the MmuC2c4 to the target gene sequence, wherein targeted binding of the MmuC2c4 endonuclease to the dsDNA results in cleavage or degradation of mRNA transcribed from the dsDNA, whilst leaving the dsDNA intact. Therefore, an important application of the products and methods of the invention herein described is for the directed silencing of gene expression by targeting RNA transcripts.

The gRNA may recognise a target sequence in dsDNA having a protospacer adjacent motif (PAM) sequence of TTM 5′, 5′ NTTM or 5′ CTM.

The MmuC2c4 RNA endonuclease preferably has an amino acid sequence as set forth in SEQ ID NO:1 or a sequence of at least 55% identity thereto.

In a method in accordance with the invention, an organism or cell is transfected with an expression vector as herein defined, and (a) further transfected with a second expression vector which comprises an expression promoter and a sequence under the control of the promoter which encodes a guide RNA (gRNA) having a nucleotide sequence which recognises a target locus of interest and the PAM sequence of TTM 5′, 5′ NTTM or 5′ CTM in dsDNA; or (b) a gRNA having a nucleotide sequence which recognises a target locus of interest and the PAM sequence of TTM 5′, 5′ NTTM or 5′ CTM in dsDNA, is introduced directly into the organism or cell.

In a method of the invention, an organism or cell is transfected with the aforementioned expression vector.

In further methods of the invention, a MmuC2c4 RNA endonuclease with an amino acid sequence as set forth in SEQ ID NO:1 or a sequence of at least 55% identity thereto is introduced into the cell, and wherein (i) a gRNA having a nucleotide sequence which recognises a target locus of interest and the PAM sequence of 5′ TTM 5′, 5′ NTTM or 5′ CTM in dsDNA, is also introduced into the organism or cell; or (ii) the organism or cell is transfected with an expression vector which comprises an expression promoter and a sequence under the control of the promoter which encodes a guide RNA (gRNA) having a nucleotide sequence which recognises a target locus of interest and the PAM sequence of 5′ TTM 5′, 5′ NTTM or 5′ CTM in dsDNA.

The MmuC2c4 RNA endonuclease may be introduced into the organism or cell substantially simultaneously, sequentially or separately from (i) or (ii).

In other methods of the invention, gRNA may be associated with the MmuC2c4 RNA endonuclease upon introduction into the organism or cell.

In another aspect in accordance with the invention, there is provided a DNA comprising a nucleotide sequence encoding a MmuC2c4 (dMmuC2c4) RNA endonuclease, optionally a catalytically inactive MmuC2c4 (dMmuC2c4) RNA endonuclease, having an amino acid sequence as set forth in SEQ ID NO:2 or a sequence of at least 55% identity thereto, wherein the endonuclease is fused to at least another protein. Such embodiments of the invention may be referred to as chimeric proteins. In particular embodiments described below such chimeric proteins may be referred to as gene editors.

That other protein may be selected from an enzyme, a ligand, a marker; optionally wherein the enzyme is a cytidine deamination enzyme and/or a uracil glycosylase inhibitor (UGI); preferably wherein the RNA endonuclease is fused to both the cytidine deamination enzyme and the uracil glycosylase inhibitor (UGI). In such aspects of the invention, the modified Mmu type I nucleases of the invention may be used for the purposes of genetic engineering through base editing.

The cytidine deamination enzyme and UGI may be fused to the N-terminal or C-terminal end of the dMmuC2c4 endonuclease; optionally wherein the cytidine deamination enzyme is fused directly to the N-terminal end of the dMmuC2c4 endonuclease, and the UGI is fused to the cytidine deamination enzyme.

The UGI may be derived from any suitable organism, e.g. E coli, or H. sapiens.

In further embodiments, the RNA endonuclease is preferably catalytically inactive for endonuclease activity; optionally wherein the RNA endonuclease comprises a D485A substitution; preferably wherein the RNA endonuclease has an amino acid sequence as set forth in SEQ ID NO: 4 or a sequence of at least 55% identity therewith.

In another aspect there is a polynucleotide comprising a nucleotide sequence encoding (i) a MmuC2c4 (dMmuC2c4) RNA endonuclease having a nucleotide sequence as set forth in SEQ ID NO:2 or a sequence of at least 55% identity therewith, optionally a catalytically inactive MmuC2c4 (dMmuC2c4) RNA endonuclease, (ii) a nucleotide sequence of a cytidine deamination enzyme, and (iii) a nucleotide sequence of a uracil glycosylase inhibitor (UGI), wherein the sequences of dMmuC2c4 RNA endonuclease, cytidine deamination enzyme and UGI are ordered so that the expression product of the polynucleotide is a fusion of dMmuC2c4 RNA endonuclease with cytidine deamination enzyme and UGI.

Also in accordance with the invention, there is an expression vector (hereinafter referred to as type A) comprising an expression promoter and (i) a dMmuC2c4 RNA endonuclease having a nucleotide sequence as set forth in SEQ ID NO:2 or a sequence of at least 55% identity therewith, (ii) a nucleotide sequence of a cytidine deamination enzyme, and (iii) a nucleotide sequence of a uracil glycosylase inhibitor (UGI), wherein the sequences of dMmuC2c4 RNA endonuclease, cytidine deamination enzyme and UGI are ordered so that the expression product of the polynucleotide is a fusion of dMmuC2c4 RNA endonuclease with cytidine deamination enzyme and UGI.

In such an expression vector the in frame reading order of the nucleotide sequences may be (i) followed by (ii) followed by (iii).

An aforementioned expression vector of the invention may further comprise a nucleotide sequence comprising an expression promoter and a sequence under the control of the promoter encoding a guide RNA (gRNA) with a nucleotide sequence which recognises a target locus of interest and the PAM sequence of 5′ TTM, 5′ NTTM or 5′ CTM in dsDNA. This is hereinafter referred to as a type B expression vector.

In any of the aspects of the invention defined above which are a RNA endonuclease, a polynucleotide or an expression vector, the cytidine deamination enzyme may be cytidine deaminase (CDA), apolipoprotein B mRNA editing enzyme (APOBEC) or activation-induced cytidine deaminase (AID). The APOBEC may be human, e.g. APOBEC1, APOBEC2, APOBEC3A, APOBEC3B, APOBEC3C, APOBEC3D, APOBEC3F, APOBEC3G, APOBEC3H or APOBEC4.

Base editors of the invention may include an LVA degradation tag (to reduce toxicity of the BE). This LVA tag may be present independently of any linker or NLS (see below).

In some embodiments, base editors of the invention may include a Nuclear Localisation Signal (NLS) as will be familiar to a person of skill in the art. An NLS may be present independently of any linker or LVA tag.

Also provided by the invention is a cell comprising an expression vector of type A as hereinbefore defined. Such cells of the invention may further comprise a second expression vector which comprises an expression promoter and a sequence under the control of the promoter which encodes a guide RNA (gRNA) having a nucleotide sequence which recognises a target locus of interest and the PAM sequence of 5′ TTM, 5′ NTTM or 5′ CTM in dsDNA.

The invention further provides a method of generating a C to T mutation or mutations at a target locus in a double stranded DNA (dsDNA) in an organism, cell or cell-free system, comprising exposing the dsDNA to a dMmuC2c4 RNA endonuclease of the invention as hereinbefore defined, and a guide RNA (gRNA) having a nucleotide sequence which recognises a target locus of interest and the PAM sequence of TTM 5′, 5′ NTTM or 5′ CTM in dsDNA.

In certain embodiments of the aforementioned method of the invention, the organism or cell may be transfected with a type A expression vector of the invention as hereinbefore defined, and (a) further transfected with a second expression vector which comprises an expression promoter and a sequence under the control of the promoter which encodes a guide RNA (gRNA) having a nucleotide sequence which recognises a target locus of interest and the PAM sequence of 5′ TTM, 5′ NTTM or 5′ CTM in dsDNA; or (b) a gRNA having a nucleotide sequence which recognises a target locus of interest and the PAM sequence of 5′ TTM, 5′ NTTM or 5′ CTM in dsDNA, is introduced directly into the organism or cell.

In other embodiments of methods of the invention, an organism or cell may be transfected with an expression vector of type B as hereinbefore defined.

In a method aspect of the invention, a MmuC2c4 RNA endonuclease with an amino acid sequence as set forth in SEQ ID NO:1 or a sequence of at least 55% identity thereto is introduced into an organism or cell, and wherein (i) a gRNA having a nucleotide sequence which recognises a target locus of interest and the PAM sequence of 5′ TTM, 5′ NTTM or 5′ CTM in dsDNA, is also introduced into the organism or cell; or (ii) the organism or cell is transfected with an expression vector which comprises an expression promoter and a sequence under the control of the promoter which encodes a guide RNA (gRNA) having a nucleotide sequence which recognises a target locus of interest and the PAM sequence of 5′ TTM, 5′ NTTM or 5′ CTM in dsDNA.

The MmuC2c4 RNA endonuclease may be introduced into the organism or cell substantially simultaneously, sequentially or separately from (i) or (ii).

In certain methods, the gRNA is preferably associated with the MmuC2c4 RNA endonuclease upon introduction of the same into the organism or cell.

The methods of the invention may be applied to eukaryotic organisms or cells. Thus, the invention includes any, animal or cell, produced by the present methods, or a progeny thereof. The progeny may be a clone of the produced plant or animal or may result from sexual reproduction by crossing with other individuals of the same species to introgress further desirable traits into their offspring. The cell may be in vivo or ex vivo in the cases of multicellular organisms, particularly animals or plants.

In any of the aforementioned methods of the invention, the organism or cell is preferably a eukaryote. However, in certain aspects, the methods of the invention may not include methods of prevention or treatment of disease when performed on the human or animal body. The invention may however include the modification of cells or tissue obtained from a human or animal which is then modified in accordance with methods of the invention. The modified tissue or cells may then be returned to the human or animal body, whether the same as from which the tissue or cells were removed, or different.

Accordingly, the invention includes any described products of Mmu nucleases, Mmu nuclease-based gene editor molecules, and ribonucleoprotein complexes of these, for use as a medicament, for the prevention or treatment of human or animal disease. For example, where gene silencing is known or suspected to offer a mode of treatment for a particular human or animal disease, then the present gene silencing aspects of the present invention may be used. Similarly, where single base change or changes offer a mode of treatment for a particular human or animal disease, then again the gene editor molecule aspects of the present invention may be used.

In other aspects, the invention provides chimeric fusion proteins of the MmuC2c4 endonuclease of the invention as hereinbefore defined, such fusions comprising the MmuC2c4 as defined herein together with another functional protein or moiety. Advantageously, the ability of the MmuC2c4 of the invention is modified thereby, for example by cleaving the target nucleic acid and/or marking it and/or modifying it. It will therefore be appreciated that additional proteins may be provided along with the MmuC2c4 protein to achieve this. Accordingly, MmuC2c4 fusion proteins of the invention may further comprise at least one functional moiety and/or may be provided as part of a protein complex comprising at least one further protein. Preferably, the at least one functional moiety or protein may be translationally fused to the Cas protein through expression in natural or artificial protein expression systems. Therefore the invention includes polynucleotides and expression vectors encoding the aforementioned fusion proteins.

Alternatively, the at least one functional moiety may be covalently linked by a chemical synthesis step to the Cas protein. Preferably, the at least one functional moiety is fused or linked to the N-terminus and/or the C-terminus of the Cas protein; preferably the C-terminus.

Desirably, the at least one functional moiety will be a protein. It may be a heterologous protein or alternatively may be native to the bacterial species from which the MmuC2c4 protein was derived. The at least one functional moiety may be a protein; optionally selected from a helicase, a nuclease, a helicase-nuclease, a DNA methylase, a histone methylase, an acetylase, a phosphatase, a kinase, a transcription (co-)activator, a transcription repressor, a DNA binding protein, a DNA structuring protein, a marker protein, a reporter protein, a fluorescent protein, a ligand binding protein, a signal peptide, a subcellular localisation sequence, an antibody epitope or an affinity purification tag.

In some particular aspects, the invention provides a MmuC2c4-FokI fusion, wherein the FokI is a naturally occurring bacterial type IIS restriction endonuclease, found in Flavobacterium okeanokoites. FokI has an N-terminal DNA-binding domain and a C-terminal non-specific DNA cleavage domain. Binding of FokI to a dsDNA via its 5′-GGATG-3′ recognition site results in DNA cleavage. Relative to the nearest nucleotide in the recognition sequence, the break in the first strand DNA is downstream 9 nucleotides and the break in the second the second strand DNA is 13 nucleotides upstream thereof. The endonuclease domain of the FokI is used in the fusion.

### BRIEF DESCRIPTION OF THE DRAWINGS

Embodiments of the invention are further described hereinafter with reference to the accompanying drawings, in which:

FIG. 1 shows the arrangement of the Type V-U1 CRISPR-Cas locus of Mycobacterium mucogenicum CCH10-A2.

FIGS. 2A-C are schematic diagrams of MmuC2c4 PAM assessment using a PAM-SCNR method.

FIG. 3 shows the results of PAM-SCNR deep sequencing results in the form of web logos for non-targeting spacers (NT) and targeting spacers (T).

FIG. 4 shows a schematic of dMmuC2c4 targeting at the promoter region of Green Fluorescent Protein (GFP).

FIG. 5 shows GFP repression of dMmuC2c4 and dFnCpf1 showed for different 5′ NTTN PAMs.

FIG. 6 is a schematic showing dMmuC2c4 targeting divergent RFP GFP (expressed on separate constitutive promoter).

FIG. 7 shows the results of GFP and RFP fluorescence targeted by dMmuC2c4 and MmuC2c4.

FIG. 8A is a schematic diagram of (d)MmuC2c4 targeting divergent RFP and GFP expressed on separate constitutive promoters.

FIG. 8B shows the results of GFP fluorescence targeted by dMmuC2c4 and MmuC2c4.

FIG. 8C shows the results of RFP fluorescence targeted by dMmuC2c4 and MmuC2c4.

FIG. 9A is a schematic diagram showing multiplexing of dMmuC2c4 and MmuC2c4 targeting divergent RFP and GFP expressed on separate constitutive promoters using a crRNA containing two spacers.

FIG. 9B shows the results of GFP fluorescence targeted by dMmuC2c4 and MmuC2c4.

FIG. 9C shows the results of RFP fluorescence targeted by dMmuC2c4 and MmuC2c4.

FIG. 10 is a schematic diagram showing a modified MmuC2c4 as a base editor.

FIG. 11A shows MmuC2c4 base editor recognizing a PAM and binding to the target strand. CDA then deaminates a cytosine C to a U.

FIG. 11B. A GFP is constitutively expressed until a premature stop codon is introduced by a targeted MmuC2c4 base editor. A C to T on position 13 causes a nonsense mutation, meaning a glutamine (CAA) codon is converted to a STOP codon (TAA).

FIG. 12 shows the target sequence for within the gfp gene for the MmuC2c4 as a base editor.

FIG. 13A is an alignment of the base edited positions of C-tiling protospacers targeted by dMmuC2c4 base editor.

FIG. 13B is fused deep sequencing data of protospacers 1, 3, 5 (blue) and 2, 4, 6 (orange). Base edited C's in percentage (y-axis) for different nucleotide position (x-axis).

FIGS. 13C to 13I show deep sequencing data of C-tiling protospacer. Base edited C's in percentage (y-axis) for different nucleotide position (x-axis).

FIG. 14 is a schematic diagram of base editors (MmuBE) for E. coli and Homo sapiens (Hsa).

FIG. 15A is a schematic diagram of GFP silencing by MmuBE.

FIG. 15B is a chart comparing GFP silencing activity of various MmuBEs.

FIG. 15C shows base editing target motifs C1, C2 and C3 consisting of a C on every first, second and third position of each trinucleotide.

FIGS. 15D & E are heat maps representing % of base edited C's using different variants of H. sapiens MmuBEs (MmuBE_H's). FIG. 16A is a diagram of the experimental set up for testing base editing in S. cerevisiae where the ADE2 gene is targeted by MmuBE.

FIG. 16B is a schematic diagram of base editing workflow for S. cerevisiae.

FIG. 16C shows the sequencing results of three MmuBE_S targets, ADE2_1, ADE2_2, ADE2_3.

FIG. 17A is a schematic diagram of a dMmuC2c4-FokI domain fusion with the protospacers facing inwards. FIG. 17B is a schematic diagram of a dMmuC2c4-FokI domain fusion with the protospacers facing outwards.

### DETAILED DESCRIPTION

Generally, the term “vector” herein refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

A plasmid may be vector in accordance with this description, which is a circular double stranded DNA loop into which additional DNA segments can be inserted, such as by standard molecular cloning techniques.

Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Some vectors are able to direct expression of genes to which they are operatively-linked. Such vectors are “expression vectors” and there will usually be regulatory elements, which may be selected on the basis of the host cells in which the expression takes place. This means the nucleic acid to be expressed is operably linked to the regulatory elements thereby resulting in expression of the nucleotide sequence whether in an in vitro transcription/translation system or in a host cell when the vector is introduced into the host cell.

Suitable regulatory elements include promoters, enhancers, internal ribosomal entry sites (IRES), and other expression control elements (e.g., transcription termination signals, such as polyadenylation signals and poly-U sequences). For more information the average skilled person would refer to, for example, in Goeddel, (1990), Gene Expression Technology in Methods in Enzymology vol 185, Academic Press. Regulatory elements include those giving direct constitutive expression in many types of host cell and those that direct expression of the nucleotide sequence only in certain cells (i.e., tissue-specific regulatory sequences).

A tissue-specific promoter directs expression primarily in a desired tissue of interest, such as blood, specific organs (e.g., liver, pancreas), or particular cell types. Regulatory elements may also direct expression in a temporal-dependent manner, such as in a cell-cycle dependent or developmental stage-dependent manner, which may or may not also be tissue or cell-type specific. Examples of promoters include pol I, pol II, pol III (e.g. U6 and H1 promoters). Examples of pol II promoters include, but are not limited to, retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-acting promoter, the phosphoglycerol kinase (PGK) promoter, and the EF1α promoter.

As well as promoters, regulatory elements may include enhancer elements, such as WPRE; CMV enhancers; the R-U5′ segment in LTR of HTLV-I; SV40 enhancer; and the intron sequence between exons 2 and 3 of rabbit β-globin. It will be appreciated by those skilled in the art that the design of the expression vector can depend on such factors as the choice of the host cell to be transformed, the level of expression desired, etc.

Methods of non-viral delivery of nucleic acids may include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration.

The invention encompasses methods of modifying a genomic locus of interest to change gene expression in a cell by introducing into the cell any of the compositions described herein. This may include medical uses in humans for therapeutic or non-therapeutic purposes. Furthermore, any of the methods described herein may be applied in vitro and ex vivo. For therapeutic purposes, these may be gene or genome editing, or gene therapy. The invention also encompasses methods of modifying genomic loci for non-medical uses in animals, plants, algae or fungi; or in prokaryotes including bacteria and archaea.

In any of the aforementioned aspects of the invention, “base pairing affinity” and “complementarity” may be used interchangeably and refer to the ability of a nucleic acid to form hydrogen bond(s) with another nucleic acid sequence by either traditional Watson-Crick base pairing or other non-traditional types. A percent identity (i.e. complementarity) in relation to a reference sequence, in the various descriptions of the invention, represents the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% identity). “Perfectly complementary” means that all the contiguous residues of a nucleic acid sequence will hydrogen bond with the same number of contiguous residues in a second nucleic acid sequence, and this is a preferred condition for antisense oligonucleotide binding to the targeting RNA which corresponds to 100% identity for a length of targeting RNA molecule which is the same length as the antisense oligonucleotide. Also, the term “substantially complementary” as used herein refers to a degree of identity that is at least 90%, 95%, 97%, 98%, 99%, or 100% between the portion of the antisense oligonucleotide and the equivalent length of targeting RNA molecule. This may also correspond to nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions surrounding the nucleic acids, temperature, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993), each of which are incorporated herein by reference. The Tm is the temperature at which more than 50% of a given strand of a nucleic acid molecule is hybridized to its complementary strand. The following is an exemplary set of hybridization conditions and is not limiting:

Very High Stringency (allows sequences that share at least 90% identity to hybridize) Hybridization: 5×SSC at 65° C. for 16 hours; wash twice: 2×SSC at room temperature (RT) for 15 minutes each; wash twice: 0.5×SSC at 65° C. for 20 minutes each.

High Stringency (allows sequences that share at least 80%> identity to hybridize) Hybridization: 5×-6×SSC at 65° C.-70° C. for 16-20 hours; wash twice: 2×SSC at RT for 5-20 minutes each; wash twice: 1×SSC at 55° C.-70° C. for 30 minutes each.

Low Stringency (allows sequences that share at least 50%> identity to hybridize); hybridization: 6×SSC at RT to 55° C. for 16-20 hours; wash at least twice: 2×-3×SSC at RT to 55° C. for 20-30 minutes each.

In terms of percentage identity characterising the extent of variation of the Mmuc2C4 or dMmuc2C4 of the invention with the specified reference sequence, the degree of identity may be any of: at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, most preferably at least 90%, at least 91%, at least 92%, at least 93%, at least 94%, at least 95%, at least 96%, at least 97%, at least 98%, or at least 99%.

In the aforementioned kits of the invention, the vectors and constructs may be as described in more detail, for example in WO2013/176772 A1 or WO2014/093595 A1, both of which are incorporated herein by reference.

In any such systems comprising vectors, the one or more vectors may comprise one or more viral vectors, such as one or more retrovirus, lentivirus, adenovirus, adeno-associated virus or herpes simplex virus. Also, in any such systems comprising regulatory elements, at least one of said regulatory elements may comprise a tissue-specific promoter, whether or animal including human, or plant.

In any of the aspects of the invention, the targeting RNA molecule is designed to have complementarity, where hybridization between a target sequence and the RNA targeting molecule promotes the formation of a RNA-targeting complex. Targeting RNA molecules in accordance with the invention may include mature crRNA, guide RNA (gRNA) or single guide RNA (sgRNA) and these terms can be used interchangeably. In general, a targeting RNA has a sufficient complementarity with the target polynucleotide sequence to hybridize with the target sequence and direct sequence-specific binding of the CRISPR enzyme or Cascade complex to the target sequence. The degree of complementarity between a targeting RNA and its corresponding target sequence may be more than about 50%, 60%, 75%, 80%, 85%, 90%, 95%, 97.5%, 99%, or more, with optimal algorithmic alignment. Throughout this specification in any context, optimal alignment may be determined using, for example, any of the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

In certain uses of the products of the invention, individual components of dMmuC2c4 and MmuC2c4 or the dMmuC2c4 gene editor, may be pre-assembled as a ribonucleoprotein (RNP) complex, this can be used to achieve the desired target locus effects. Such RNPs may be introduced directly into cells for example by electroporation or by bombardment using RNP-coated particles; also chemical transfection or by some other means of transport across a cell membrane.

In accordance with the invention, there are two principle approaches being described. Firstly the silencing approach whereby nascent or would-be RNA transcripts are targeted such that they are inhibited partially or fully from forming. Secondly, the base editing whereby dsDNA is targeted and modifications are introduced into the DNA sequence by enzymatically mediated chemical changes to the nucleotide residues. In connection with the first mode, where an RNP complex is introduced into cells it is only expected to work to silence genes by preventing or inhibiting RNA transcription for a short time, usually a few hours to a few days. For a longer silencing effect then a person of average skill will understand that the Mmu nuclease enzymes of the invention will need to be introduced into the cell as DNA which can be transcribed and translated so as to provide the Mmu nucleases in the cell. A stable or replicating plasmid can be used, or the Mmu nuclease DNA sequence can be integrated into the genome of the cell concerned, whenever possible at a defined position so that it is not likely to have deleterious effects.

For the second mode of base editing, all possible delivery modes, as also described herein, may be used.

The endonuclease or gene editor may be introduced into a cell separately, simultaneously or sequentially with an isolated targeting RNA, usually in the form of a gRNA.

Base editing in the context of the present invention using MmuC2C4 or dMmuC2C4 involves site-specific modification of the DNA base along with manipulation of the DNA repair machinery to avoid faithful repair of the modified base. The base editors of the invention are chimeric proteins composed of the MmuC2C4 or dMmuC2C4 (together with targeting RNA to form an RNP) and a catalytic domain capable of deaminating a cytidine or adenine base. Advantageously, using the dMmuC2C4 of the invention there is no generating of DSBs giving rise to insertions and deletions (indels) at target and off-target sites.

Hydrolytic deamination of adenosine (A) and cytidine (C) into inosine (I) and uridine (U) means these are read as guanosine (G) and thymine (T), respectively, by polymerase enzymes. The conversion of C into U might result in the onset of base excision repair, where a U from the DNA is excised by uracil DNA N-glycosylase (UNG). This is followed by a repair into C through error-free repair or error-prone repair that results in base substitutions. Blocking the base excision is promoted by the use of uracil DNA glycosylase inhibitor (UGI).

Cytidine deaminase-based DNA base editors catalyze the conversion of cytosine into uracil, for example APOBEC deaminase which converts cytidine into thymidine. In the base-editing system, APOBEC, guided by dCas9, deaminates a specific cytidine to uracil; the resulting U-G mismatches are resolved via repair mechanisms and form U-A base pairs, and subsequently T-A base pairs. Thus, these base editors can be used to produce C-to-T point mutations (in dsDNA: C-G to T-A).

Cytidine deaminase converts C into U and subsequently uracil DNA glycosylase can perform error-free repair, converting the U into the wild-type sequence. The addition of the UGI inhibits the base excision repair pathway, resulting in a three-fold increased efficiency.

Multiple additional base-editing systems can be made in accordance with the invention, with different deaminases. For example, activation-induced cytidine deaminase (AID); optionally with UGI is similar. Because the activity of the UGI inhibits excision repair and improves the base-editing efficiency, two UGI molecules can be included; e.g. one at the C- and one at the N-terminus.

In terms of what determines the best base editor for a given application, the choice of base editor will depend on the availability of a PAM sequence, the presence of a C nucleotide relative to the PAM, and how the base-editor reagents are delivered to the target cell. Furthermore, the nature of the edits could also be determined by the base editor.

Adenine base editors may be made in accordance with the invention to modify adenine bases. The deamination of adenosine yields inosine, which can base pair with cytidine and subsequently be corrected to guanine, thereby converting A into G, or A-T into G-C

In summary, base editors using cytosine deaminases can convert C-G via U-G into T-A, and adenosine deaminases can convert A-T via I-C into G-C. These base modifications can generate targeted sequence variation in a precise manner.

In accordance with the invention, a base editor may comprise a linker which is comprised, or consists of, a number of amino acids. The length of the linker may be selected from a number of amino acids consisting of: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 and 200.

Linkers may be used as a way of modifying or varying the editing window of the base editors of the invention. Such modifications will be apparent to a person of skill in the art and having reference to the accompanying examples.

Also in accordance with the invention are ADAR2-based RNA base editors. Very recently, RNA base editors have been developed and used to modulate biological processes. Several systems, including ADAR2, deaminate adenosine to inosine, which is read as guanine by the translational machinery, have been used for RNA editing.

Methods of the invention may be in vitro, for example they are performed using a synthetic mix of the reaction components in a suitable buffer system. In some in vitro embodiments there is used a cell-free transcription/translation system.

Methods of the invention may be employed occurring ex vivo, for example in a cell or cell culture. In ex vivo treatments, diseased cells are removed from the body, treated with the products of the invention, or the base editor of the invention, and then transplanted back into the patient. Ex vivo editing has an advantage of allowing the target cell population to be well defined and the specific dosage of therapeutic molecules delivered to cells to be specified. In one aspect, the invention provides therapeutic methods for organisms (humans or animals), whereby a single cell or a population of cells is sampled or cultured and then that cell or cells are modified ex vivo, as described herein, and then re-introduced into the organism. The cells modified ex vivo may be stem cells, whether embryonic or induce pluripotent or totipotent stem cells, including totipotent stem cells, which may preferably be non-human totipotent stem cells.

In vivo embodiments are also provided. In vivo editing can be used advantageously from this disclosure and the knowledge in the art.

The base editing tool of the invention based on the described MmuC2c4 or the dMmuC2c4 gene editors are smaller than Cas9 or Cas12a. Therefore a ribonucleoprotein complex (RNP) formed of the Mmu nucleases of the invention, including the gene editor versions, and an RNA guide (gRNA) is advantageously directly introduced into cells. Alternatively, the Mmu nuclease of the invention or the gene editor versions and the gRNA may be introduced independently into cells, that is to say simultaneously, separately ro sequentially. Such introduction may be by microinjection into the nucleus or cytoplasm of a cell or by other means. For comprehensive reviews about procedures for getting proteins, gRNA or RNPs in the context of this invention, see Marschall A L J, Frenzel A, Schirrmann T, et al. “Targeting antibodies to the cytoplasm” mAbs. (2011) 3:3-16; Gu Z, Biswas A, Zhao M, Tang Y “Tailoring nanocarriers for intracellular protein delivery” Chem. Soc. Rev. (2011) 40:3638-3655. Du J, Jin J, Yan M, Lu Y “Synthetic nanocarriers for intracellular protein delivery” Curr. Drug Metab. (2012) 13:82-92.

Various physical methods of disrupting the cell membrane are useful, such as microinjection and electroporation (see Zhang Y, Yu L-C. “Microinjection as a tool of mechanical delivery” Curr. Opin. Biotechnol. (2008) 19:506-510) have been proposed for delivering compounds ranging from small molecules to proteins. Sharei A, Zoldan J, Adamo A, et al. “A vector-free microfluidic platform for intracellular delivery” Proc. Natl. Acad. Sci. (2013) 110: 2082-2087 describes a microfluidic device that transiently disrupts the plasma membrane through physical constriction. Silicon “nanowires” that pierce the cell membrane have also been reported Shalek A K, Robinson J T, Karp E S, et al. “Vertical silicon nanowires as a universal platform for delivering biomolecules into living cells” Proc. Natl. Acad. Sci. (2010) 107:1870-1875.

There are also peptide-based strategies using cell penetrating peptides (CPP) which can enhance permeability of the endonucleases, RNPs and base editors of the invention. For example the TAT peptide can be covalently coupled. Also, an amphiphilic CPP Pep-1 can noncovalently complex and translocate peptide and protein cargos Morris M C, Depollier J, Mery J, et al. “A peptide carrier for the delivery of biologically active proteins into mammalian cells” Nat. Biotechnol. (2001) 19: 1173-1176.

There is also for example substance P (SP), an 11-residue neuropeptide which can be conjugated to the products of the invention (Harford-Wright E, Lewis K M, Vink R, Ghabriel M N. “Evaluating the role of substance P in the growth of brain tumors” Neuroscience (2014) 261: 85-94.

There are also various pore- or channel-forming proteins of bacterial origin which may be used to translocate proteins and RNPs of the invention into cells. Chatterjee S, Chaudhury S, McShan A C, et al. “Structure and biophysics of type Ill secretion in bacteria. Biochemistry (Mosc)” (2013) 52: 2508-2517 teaches a sophisticated secretion system which transport proteins directly from the bacterial cytoplasm to the eukaryotic host. Doerner J F, Febvay S, Clapham D E. “Controlled delivery of bioactive molecules into live cells using the bacterial mechanosensitive channel MscL” Nat. Commun. (2012) 3: 990 describes functional expression of an engineered bacterial channel (MscL) in mammalian cells, the opening and closing of which could be controlled chemically. Alternatively, the cholesterol-dependent cytolysin (CDC) family of pore-forming toxins, which are capable of forming macropores up to 30 nm in diameter may be useful as “reversible permeabilization” reagents for delivering proteins or RNPs of the invention. (See Dunstone M A, Tweten R K. “Packing a punch: the mechanism of pore formation by cholesterol dependent cytolysins and membrane attack complex/perforin-like proteins” Curr. Opin. Struct. Biol. (2012) 22: 342-349; Provoda C J, Stier E M, Lee K-D. “Tumor cell killing enabled by listeriolysin O-liposome-mediated delivery of the protein toxin gelonin.” J. Biol. Chem. (2003) 278: 35102-35108; and Pirie C M, Liu D V, Wittrup K D. “Targeted cytolysins synergistically potentiate cytoplasmic delivery of gelonin immunotoxin” Mol. Cancer Ther. (2013) 12: 1774-1782.

In addition to pore- or channel-forming proteins, the membrane-translocating domains of bacterial toxins have been proposed as a modular tool that can be fused to, and enhance the intracellular delivery of, other proteins (see Sandvig K, van Deurs B. “Membrane traffic exploited by protein toxins” Annu. Rev. Cell. Dev. Biol. (2002) 18: 1-24; Johannes L, Römer W. “Shiga toxins—from cell biology to biomedical applications” Nat. Rev. Microbiol. (2010) 8: 105-116.

Additionally, Lawrence M S, Phillips K J, Liu D R. “Supercharging proteins can impart unusual resilience” J. Am. Chem. Soc. (2007) 129: 10110-10112 provides “supercharged” GFP, a variant engineered to have high net positive charge (+36), and certain human proteins with naturally high positive charge (see Cronican J J, Thompson D B, Beier K T, et al. “Potent delivery of functional proteins into mammalian cells in vitro and in vivo using a supercharged protein” ACS Chem. Biol. (2010) 5: 747-752; or Cronican J J, Beier K T, Davis T N, et al. “A class of human proteins that deliver functional proteins into mammalian cells in vitro and in vivo” Chem. Biol. (2011) 18: 833-838 have been reported to translocate across the cell membrane.

There are also virus-based strategies for packaging proteins and RNPs of the present invention into virus-like particles (see Kaczmarczyk S J, Sitaraman K, Young H A, et al. Protein delivery using engineered virus-like particles. Proc. Natl. Acad. Sci. (2011) 108: 16998-17003) or attaching them to an engineered bacteriophage T4 head (see Tao P, Mahalingam M, Marasa B S, et al. “In vitro and in vivo delivery of genes and proteins using the bacteriophage T4 DNA packaging machine” Proc. Natl. Acad. Sci. (2013) 110: 5846-5851) has been reported to enhance cytosolic delivery.

Further, there are lipid and polymer-based strategies. The proteins or RNPs of the invention may be encapsulated in liposomes (see Torchilin V. Intracellular delivery of protein and peptide therapeutics. Drug Discov Today Technol. (2008) 5:e95-e103) or complexed with lipids. Regarding the latter strategy, lipid formulations that have been successful in the transfection of DNA may be used. For example, a formulation based on a mixture of cationic and neutral lipids.

Similarly, polymer-based formulations that have been successfully used for nucleic acid transfections have also been examined for their ability to “transfect” proteins. For example, polyethylenimine (PEI) or poly-β-amino esters (PBAEs) which may be in the form of biodegradable nanoparticles.

Also inorganic material-based strategies may be used; for example including silica, carbon nanotubes, quantum dots, or gold nanoparticles.

Another method is available called induced transduction by osmocytosis and propanebetaine ((iTOP) (see D′Astolfo, D. S. et al. Efficient intracellular delivery of native proteins. Cell 161: 674-690 (2015). This method allows efficient delivery of CRISPR-Cas9 into a wide variety of primary cell types. The iTOP approach enables virus-free transduction of native proteins and does not rely on additional peptide tags, which may interfere with protein function or editing efficiency and is particularly effective for transduction of cell types that are refractory to other delivery methods. For more information see Wen Y. Wu (2018) Nature Chem Biol. 14: 642-651.

The invention therefore includes kits for gene editing comprising one or more containers comprising a ribonucleoprotein complex comprising an endonuclease or gene editor of the invention and a targeting RNA molecule. In other aspects, a kit of the invention comprises one or more containers comprising in one container (a) an endonuclease or a gene editor of the invention; and in another container (b) a targeting RNA molecule.

Kits of the invention may comprise instructions for operation and use, wherein such instructions can be in the form of accompanying leaflet in a package comprising the kit components and/or the instruction materials can be available in any format online.

The kits may also include additional components to assist with sample preparation such as buffers or reagent mixes. Additionally or alternatively kits may include additional components to assist in the transfection of vectors into cells or the direct take up of oligonucleotides into cells.

In some aspects of the kit or synthetic composition of the invention, there may be a Nuclear Localisation Signal (NLS) in proximity to the N- or C-terminus of the endonuclease, gene editor or RNP of the invention. This naturally targets the same to the nucleus of a eukaryotic cell.

### EXAMPLES

Materials and Methods

Strains, Plasmids and Growth Conditions

E. coli DH5-α and DH10-β strains were used for plasmid construction. The plasmids used for this study followed the PAM-SCNR system, which consists of three plasmids (see Leenay, Ryan T., et al. (2016) “Identifying and visualizing functional PAM diversity across CRISPR-Cas systems.” Molecular Cell 62.1: 137-147.

The first plasmid is a pBAD33 containing Cas proteins under expression control of a constitutive J23108 promoter (pCas). The second plasmid is a pBAD18 expressing CRISPR-arrays under a constitutive J23119 promoter (pCRISPR) and the third plasmid is pAU66 target plasmid containing a protospacer and the PAM-SCNR circuit (pPAM-SCNR). All experiments were carried out in E. coli BW25113 containing knockout in laci, lacz and the type I-E CRISPR-Cas operon. All E. coli strains were grown at 37° C. and 220 RPM in Luria Bertani medium (LB). Antibiotics such as chloramphenicol (25 μg/ml), ampicillin (50 μg/ml) and kanamycin (50 μg/ml) were added to the medium when appropriate.

Plasmid Construction

All pCas plasmids were constructed by Gibson assembly. Catalytically inactive MmuC2c4 was generated by mutating D485A. pCRISPR plasmids were initially constructed by digestion and ligation. An initial PCR was performed to introduce restriction sites (KpNI upstream and BbsI downstream) and a CRISPR repeat in the amplified vector. The digested vector was then ligated to a spacer-repeat sequence, which was generated by oligo annealing. The overhang flanking the spacer and repeat sequence complements the overhangs generated by BbsI and KpnI, respectively. pPAM-SCNR plasmids containing different PAMs were constructed by site directed mutagenesis. pPAM-SCNR was amplified at the pLacIq and GFP region, followed by BamHI digestion and ligation to construct pLacIq-GFP. pLacIq-GFP was then digested with BamHI followed by ligation with an mRFP containing compatible overhangs.

### PAM-SCNR Assay

Cells containing both pCas and pCRISPR plasmids were made chemically competent and were transformed with the PAM Library. After the recovery step, the recovery culture was inoculated in 10 ml LB medium (1:100) and grown overnight. The overnight culture was then used to inoculate 10 ml LB medium (1:100) containing various concentration (0, 10, 1000 μM) of IPTG and cultured to an OD₆₀₀ of about 0.5 followed by flow cytometry analysis and cell sorting. Green fluorescent cells were sorted using a Sony SH800S cell sorter containing a 70 μM nozzle chips. Sorted cells were grown in 10 ml LB medium overnight. Plasmids were extracted from the pre- and post-sorted samples and send for deep-sequencing for PAM assessment.

### Base Editing Assay

BW25113 cells were co-transformed with pCas-BaseEditor, pCRISPR and pLacIq-GFP plasmids. Colonies were inoculated in LB medium and grown for three days with re-innoculation into fresh medium every 24 hours. After three days, cells were plated and screened for non-fluorescent colonies. These colonies were then selected for a colony PCR, amplifying the GFP region of the plasmid, followed by Sanger sequencing.

### Example 1: Characterisation of Type V-U1 System from the Bacterium Mycobacterium mucogenicum CCH10-A2 (Mmu)

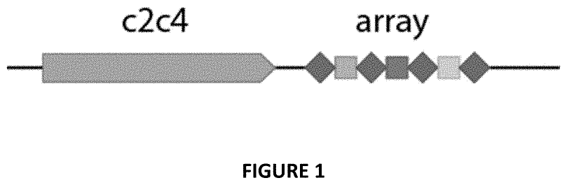

FIG. 1 shows how the CRISPR-Cas locus consists of a Cas nuclease (C2c4, or Type V-U1) and a long CRISPR array.

In vitro, the inventors have demonstrated pre-crRNA processing (Repeat-Spacer-Repeat) in case of the analogous type V-U1 system from the bacterium Clostridium bacterium DRI-13 (CbaC2c4). Attempts with the MmuC2c4 nuclease showed similar trends.

### Example 2: In Vivo PAM-Assessment

MmuC2c4 PAM assessment utilizes the PAM-SCNR method, as previously described (Leenay et al., (2016) supra).

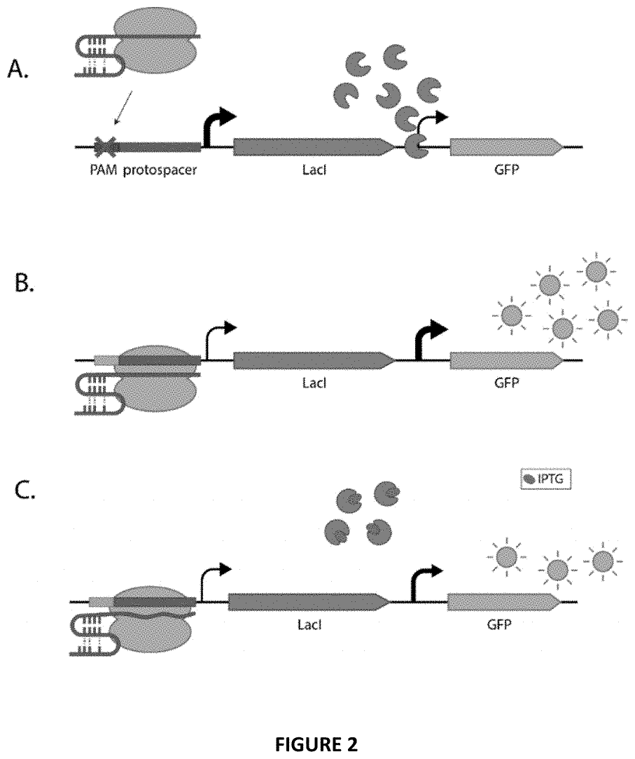

The PAM-SCNR consist of three plasmids, pCas, pCRISPR and pPAM-SCNR. pCas contains a catalytically inactive variant of the to-be-tested nuclease, in this case MmuC2c4, which we termed dead MmuC2c4 (dMmuC2c4). pCRISPR contains the CRISPR-array from M. mucogenicum. pPAM-SCNR is a PAM library consisting of a variable region (5′-NNNN) where the PAM should be located, directly upstream the protospacer; in addition, it contains a genetic circuit that expresses GFP upon PAM recognition and target binding. The circuit functions by having GFP under a Lac promoter (Plac) and Plac is constantly inhibited by Lad, the expression of which is controlled by a constitutive promoter (FIG. 2A). dMmuC2C4 targets the constitutive promoter of LacI; hence, upon PAM recognition and target binding, LacI is repressed, leading to GFP production (FIG. 2B). Positive fluorescent cells will be screened and a PAM can be determined. However, CRISPR nucleases often have several different PAMs it can recognize. Favourable PAMs lead to strong biding, whereas unfavourable PAMs lead to weaker binding. In order to asses weaker PAMs, IPTG (0, 10, 1000 μM) was added to the growth medium to control stringency. IPTG binds to LacI and hinders its inhibitory function. Less LacI in the environment means weak PAMs, thus weak binding can still cause acceptable LacI inhibition to have a GFP expression. Whereas in the absence IPTG, this weak PAM would have not caused GPP expression. Higher IPTG concentration means lower stringency. In FIG. 2 the arrow thicknesses represent promoter strength. In FIG. 2A MmuC2C4 does not recognize the PAM and protospacer. LacI is expressed and inhibits GFP expression. In FIG. 2B MmuC2c4 recognizes a PAM and binds to the protospacer, causing LacI repression, leading to GFP expression.

Three stringency conditions were tested (0, 10, 1000 μM IPTG), which corresponds to strong, medium and weak PAMs being present in the data set. The PAM-assessment was done in biological duplicates and a non-targeting spacer was used a negative control. Deep sequencing results revealed a strong 5′ NTTM (M=C/A) PAM for both replicates and all three stringency conditions. FIG. 3 shows the results of PAM-SCNR deep sequencing results in the form of web logos for non-targeting spacers (NT) and targeting spacers (T). Assay was done in biological replicates (BR1 and BR2). Y-axis shows the bit-score and why axis shows the 4 position of the NNNN PAM-library, known as the -4,-3,-2,-1 position. It is also observed that the second T is shown to be the most stringent, since the first T reduces in occurrence as IPTG concentration increases. Hence, a NCTM PAM can be considered a weak PAM.

Catalytically active MmuC2c4 was used to test a CTTA PAM and no dsDNA cleavage was observed in vivo or in vitro.

### Example 3: PAM Validation

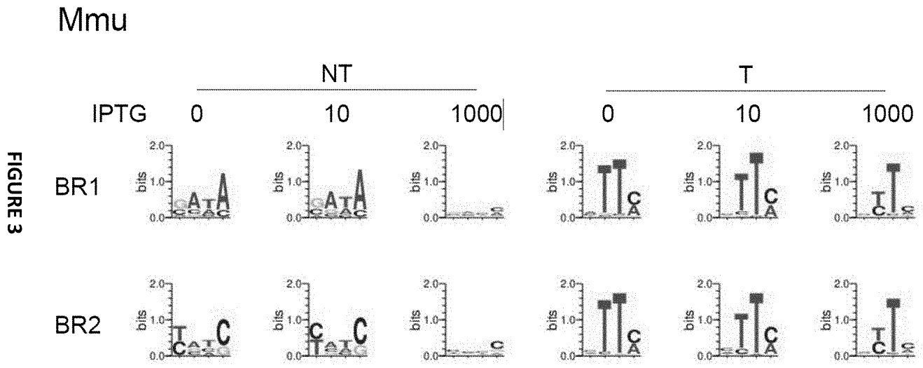

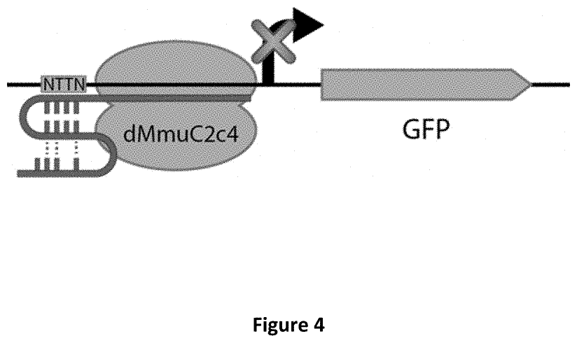

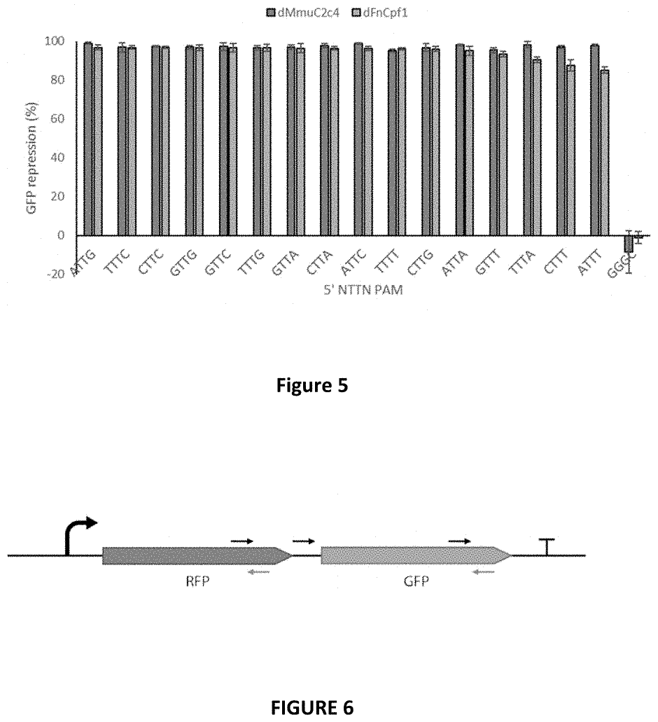

Deep sequencing results (FIG. 3) showed that dMmuC2c4 recognizes a 5′ NTTM PAM. To validate this, several 5′NTTN PAMs were tested individually. dMmuC2c4 targeted the promoter region of a constitutively expressed GFP and repression was measured by comparing GFP fluorescence of targeted and non-targeting PAMs. A schematic diagram is shown in FIG. 4. dMmuC2c4 was tested against FndCpf1 for comparison, since FndCpf1 also have a 5′NTTN. FIG. 5 shows the results and wherein in FIG. 5, PAMs are ordered from highest to lowest repression for FndCpf1 as comparison. Error bars calculated using biological replicates (n=3). From FIG. 5, it is found that MmudC2c4, similarly to FndCpf1, recognizes and binds to a protospacer containing a 5′ NTTN PAM.

### Example 4: In Vivo Local mRNA Collateral Cleavage

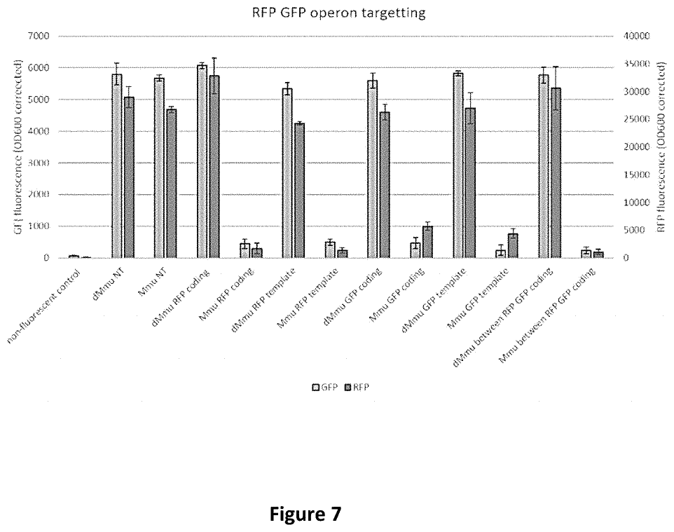

Since DNA cleavage was not observed, the following step was to asses RNA cleavage. MmuC2c4, both dead and catalytically, targeted a RFP-GFP operon controlled by a constitutive promoter. Spacers were design to target the end of the RFP and GFP coding region to minimize transcription inhibition caused by dMmuC2c4. In addition, spacers also targeted both the coding and template strand to determine strand bias. FIG. 6 shows GFP and RFP targeting by MmuC2c4 and dMmuC2c4. More particularly, FIG. 6 shows RFP-GFP operon with protospacers indicated by black (pointing to the right) and blue (pointing to the left) arrows, targeting the coding and template strand, respectively. FIG. 7 GFP (left vertical-axis) and RFP (right vertical-axis) shows fluorescence for different target sites on the RFP-GFP operon. Error bars were calculated using biological triplicates.

Then RFP and GFP fluorescence were measured to determine mRNA cleavage and/or degradation (FIG. 7). The presence of MmuC2c4, very low RFP and GFP fluorescence was observed, while on the contrary dMmuC2c4 showed high levels of fluorescence similar to the non-targeting negative control. Fluorescence drop was shown for both template and coding strand spacers, meaning no strand bias is observed. In addition, no cell viability loss were found, indicating local collateral mRNA damage.

Other Cas proteins, either bind and cleaves the same target and/or does collateral damage. A summary is provided in the table below.

| | | | Target | Target | | Size |
|---|---|---|---|---|---|---|
| Protein | tracrRNA | PAM | Binding | cleavage | collateral | (aa) |
| Cas12a | No | TTTN | dsDNA | dsDNA, | ssDNA | 1307 |
| | | 5′ | | double | | |
| | | | | stranded | | |
| | | | | break | | |
| Cas12c | Yes | TN | dsDNA | dsDNA, | — | 1209- |
| | | | | double | | 1330 |
| | | | | stranded | | |
| | | | | break | | |
| | | — | ssDNA/ | ssDNA/ | ssDNA | |
| | | | ssRNA | ssRNA | | |
| Cas12i | No | TTN | dsDNA | dsDNA, | | 1033- |
| | | | | low double | | 1093 |
| | | | | stranded | | |
| | | | | break, | | |
| | | | | prodominent | | |
| | | | | nicking on | | |
| | | | | non-target | | |
| | | | | strand | | |
| | | — | ssDNA | ssDNA | ssDNA | |
| Cas12g | yes | — | ssRNA | ssRNA | ssDNA/ | 720- |
| | | | | | ssRNA | 830 |
| Cas14a/ | yes | — | ssDNA | ssDNA | ssDNA | 529 |
| 12f | | | | | | |
| MmuC2c4 | no | TTM | dsDNA | no | ssRNA | 597 |
| | | 5′ | | | | |

### Example 5: Collateral Damage—(d)MmuC2c4 Targeting Divergent RFP GFP

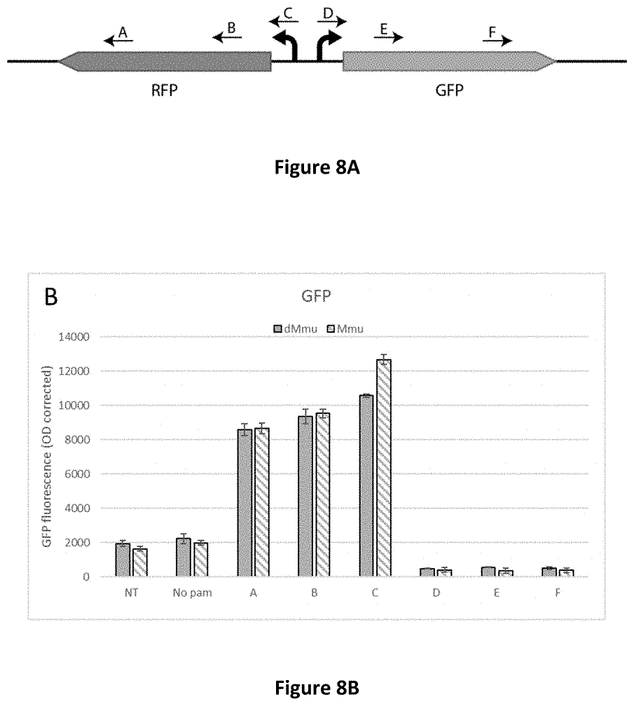

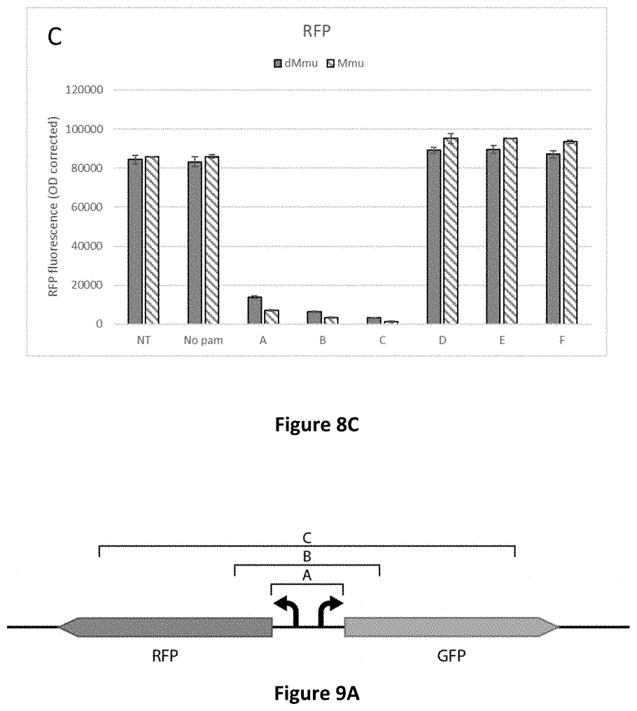

In order to verify local collateral mRNA cleavage. dMmuC2c4 targeted either RFP or GFP under control of different promoters, Ptaq and PlacIq, respectively (FIG. 8A). The arrow in FIG. 8A indicates targeted region by (d)MmuC2c4.

The results are shown in FIGS. 8B and 8C and in which NT=Non-targeting spacer; No PAM=Spacer targeting protospacer containing a GGGC PAM; and error bars are calculated using biological replicates (n=3). A fluorescence drop was detected only for the targeted fluorescent protein, whereas fluorescence for the non-targeted protein remain the same or higher. When RFP is targeted, GFP fluorescence is found to be higher when compared to its non-targeted (NT) and No PAM control. This is caused by the expression stress relieved when RFP expression is inhibited, leading to more transcription and translation components available for GFP expression. However that is not the case for RFP, when GFP is targeted. RFP fluorescence remains equal to the non-targeted (NT) and no PAM control, because RFP expression is already really high when not targeted.

### Example 6: (d)Mmuc2c4 Multiplexing

MmuC2c4 was shown previously to be able to process its own crRNA into mature crRNA, meaning MmuC2c4 should be able to target multiple genes with one crRNA. Therefore, multiplexing of the (d)Mmuc2c4 on the divergent RFP GFP construct was tested.

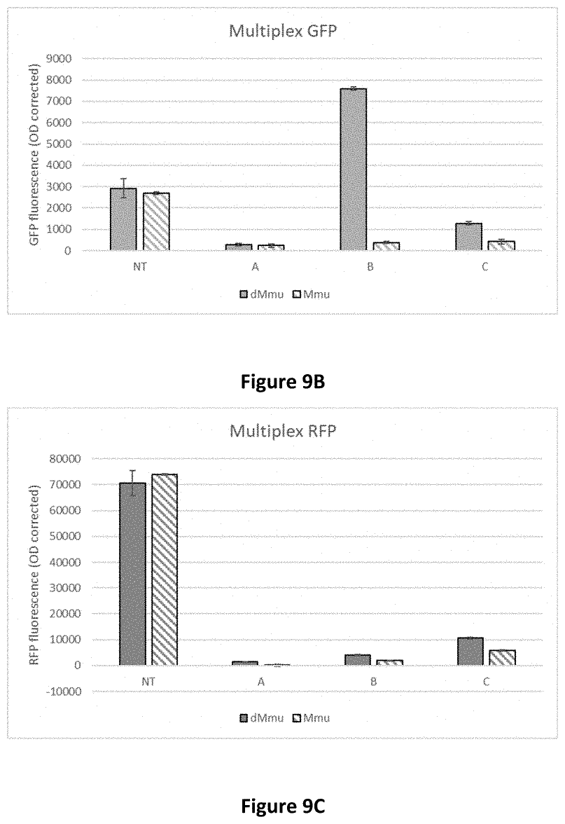

FIG. 9A is a schematic diagram showing multiplexing of dMmuC2c4 and MmuC2c4 targeting divergent RFP and GFP expressed on separate constitutive promoters using a crRNA containing two spacers. Lines indicate the two targeted regions in both GFP and RFP by (d)MmuC2c4. FIG. 9B shows the results of GFP fluorescence targeted by dMmuC2c4 and MmuC2c4. NT=Non-targeting spacer. Error bars calculated using biological replicates (n=3). FIG. 9C shows the results of RFP fluorescence targeted by dMmuC2c4 and MmuC2c4. NT=Non-targeting spacer. Error bars calculated using biological replicates (n=3).

### Example 7: Base Editing Tool Using dMmuC2c4

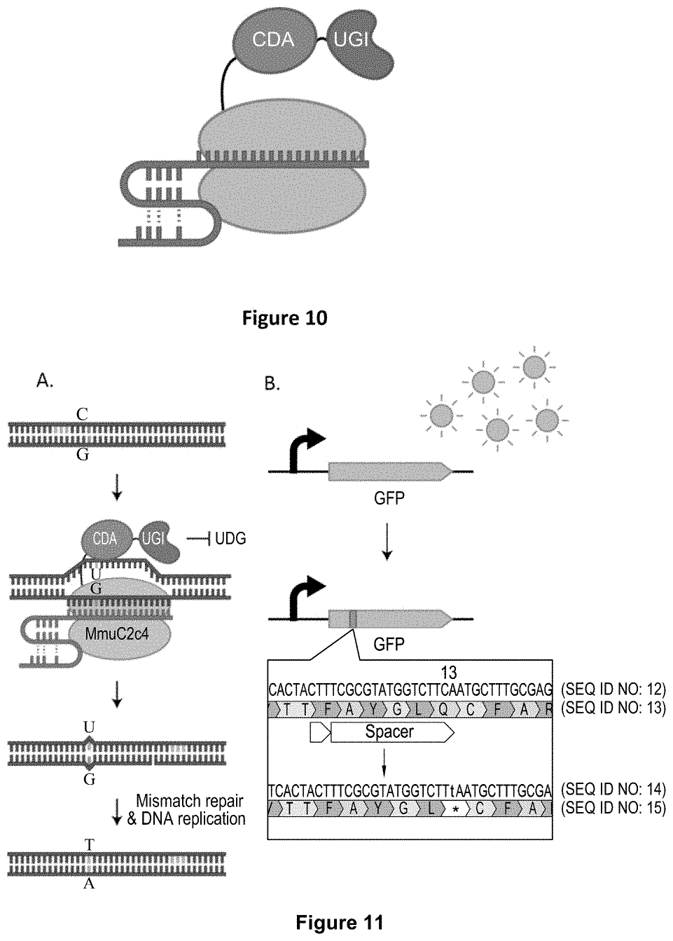

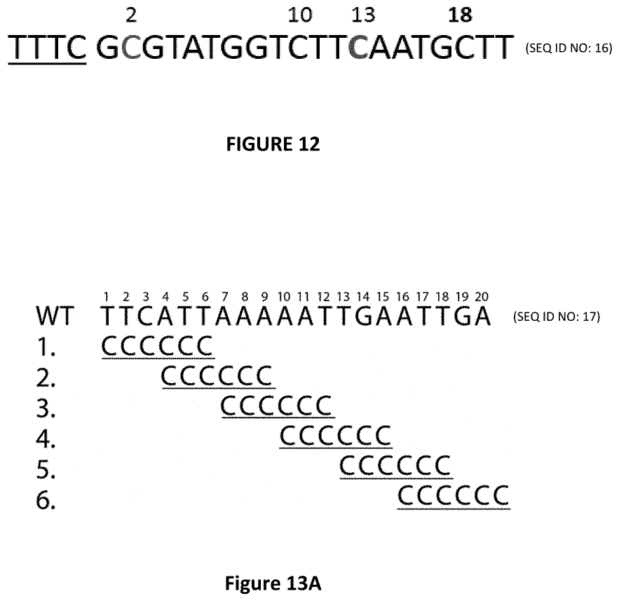

While characterizing MmuC2c4, the inventors also created a tool using dMmuC2c4, more specifically a base editor. dMmuC2C4 was fused to a cytidine deaminase (CDA) and a uracil glycosylase inhibitor (UGI). CDA deaminates cytosine (C) to uracil (U), which gets repaired to thymine (T) and UGI inhibits uracil repair. Fusing these three proteins together leads to a tool that specifically and efficiently generates C to T mutations. A MmuC2C4 base editor was created by fusing a CDA and UGI, to the N-terminal end of dMmuC2C4 (FIGS. 10 and 11A) (see Banno, Satomi, et al. “Deaminase-mediated multiplex genome editing in Escherichia coli” Nature microbiology 3.4 (2018): 423). The MmuC2c4 base editor was tested by targeting a sequence within the gfp gene. As shown in FIG. 12, the protospacer contains a C on position 2, 10, 13, 13 and 18, where position 1 is the first nucleotide of the protospacer on the 5′ end. The C on position 13 (C13) is part of a CAA codon, which codes for a glutamine. A or C to T mutation leads to a TAA pre-mature STOP codon, which terminates GFP expression.

Cells containing the MmuC2c4 base editor, the CRISPR-array and the GFP target plasmid were grown for three days. Cultures were re-inoculated and measured daily. Cultures were plated on day 3 and non-fluorescent colonies were picked and send for sequencing. There were no non-fluorescent colonies on the control plates, which was a dMmuC2C4 base editor with a non-targeting CRISPR-array. 11 colonies were sequenced and only C to T mutations were found within the target sequence. No other mutations were found in the rest of the GFP gene that was sequenced. As noted in Table 1 below, C to T mutations mostly occurred on C2 (C on position 2 of the protospacer) and C13.

TABLE 1
PAM found during sequencing of green fluorescent
colonies using the PAM-SCNR

| Amount of | |
|---|---|
| colonies | C that was mutated |
| 7 | C2 |
| 1 | C13 |
| 1 | C2 and C13 |
| 2 | C2 (mixed peaks of C and T during |
| | sequencing) |

### Example 8: Base-Editing Window Using dMmuC2c4 Base Editor

The above examples show that C to T base editing is possible with MmuC2c4. This example defines the base editing positions which are most efficient. Such positions are termed the base editing “window”. In order to find the editing window, a catalytically inactive MmuC2c4 (termed dead MmuC2c4 (dMmuC2c4)) was fused to a 121-amino acid linker, a cytidine deaminase protein CDA (to deaminate C to U), a uracil glycosylase inhibitor UGI (to inhibit repair of uracil glycosylase), and an LVA degradation tag (to reduce toxicity of the BE). This construct is termed “MmuBE_E1”, based on the nomenclature of the prokaryotic Cas9 base editors already known in the art.

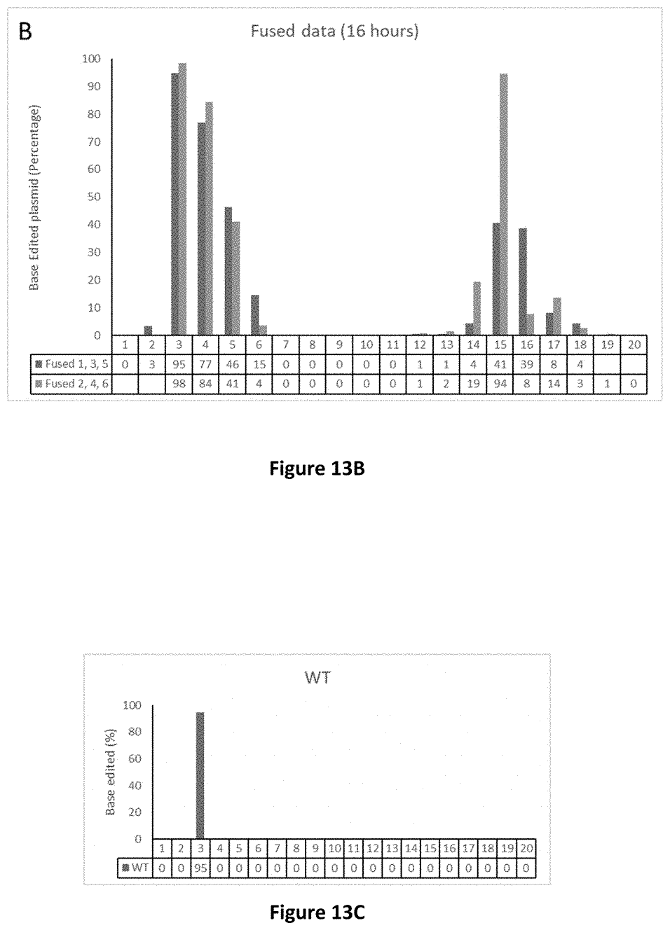

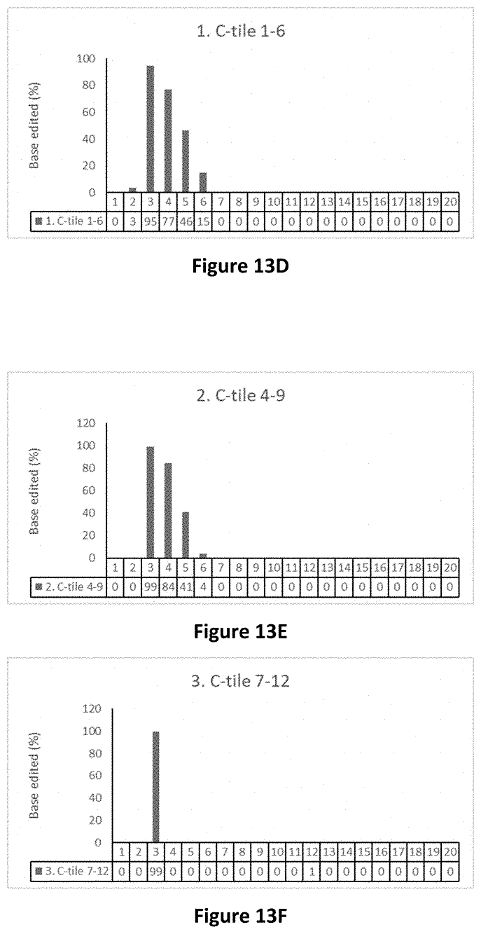

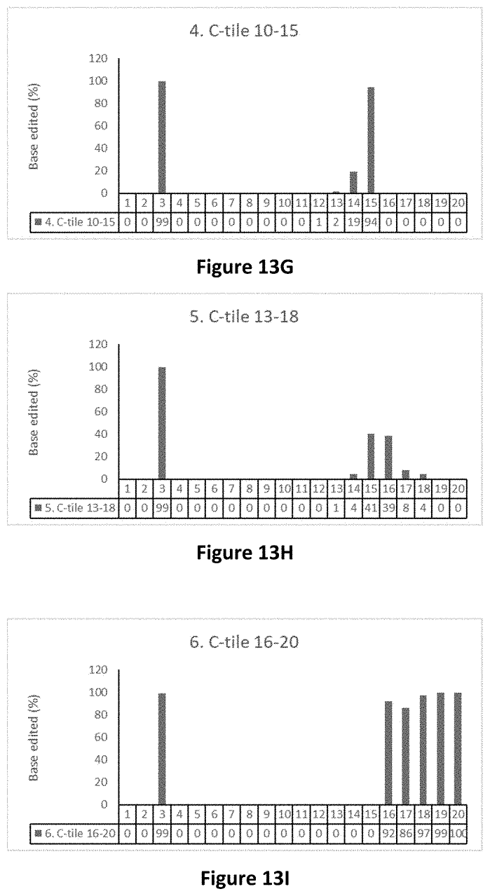

MmuBE_E1 was used to target a protospacer containing six consecutive C's e.g. positions 1-6. Different variants of this protospacer were tested, where the C's would shift three nucleotides in position towards 3′ end (C-tiling) (FIG. 13A). Protospacers 1, 3, 5 and 2, 4, 6, were chosen to be fused together, because they contain no overlapping C sequence (FIG. 13A). Cultures were grown for 16 hours and send for deep sequencing. A fused data set of all the C-tiling protospacer tested can be found in FIG. 13B, whereas data for individual samples can be found in FIG. 13C-13I. MmuBE_E1 was found to catalyze base editing in E. coli, with edits in two different regions within the target/protospacer, a PAM-proximal (positions 2-5) and a PAM-distal (positions 13-19). This implies a distinct base editing window with edits in two separated regions compared to either of Cas12a or Cas9 base editors, which introduce base edits in a single region.

### Example 9: Base-Editing Window Using dMmuC2c4 Base Editor

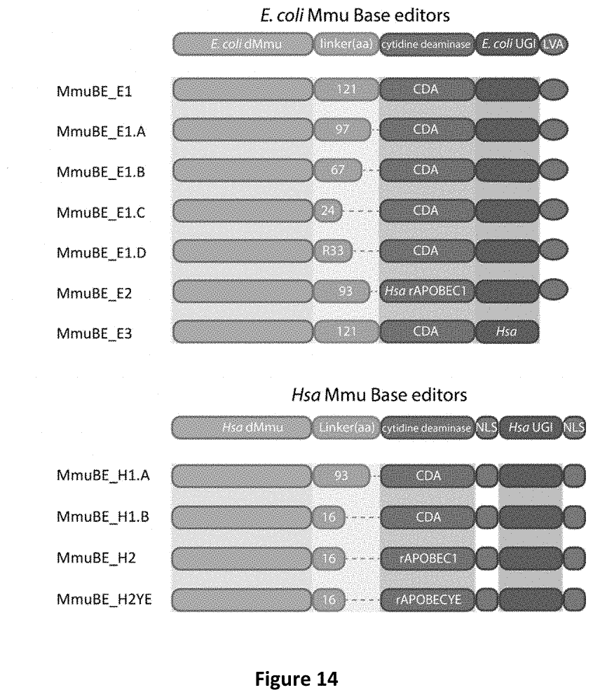

New MmuBE variants were constructed in order to change the base editing window. Various MmuBEs were designed mainly by varying the deaminase module as well as the linker length (see FIG. 14). Linker variation consisted of trimming down the flexible linker found in MmuBE_E1 from 121 to 97, 67 and 29 amino acids (aa) respectively. In addition, a rigid linker of 33 amino acids (R33) was also tested. Such rigid linkers are described in J. Tan, et al., (2019) Nature Communications 10, 1-10.

Varying linkers in MmuBE_E1 were named MmuBE_E1.A-D (FIG. 14). In addition, E. coli optimized CDA or UGI genes were substituted by either human optimized rAPOBEC1 or UGI, creating MmuBE_E2 and MmuBE_E3, respectively (FIG. 14). Next to creating E. coli MmuBEs, several MmuBEs were also constructed for mammalian cells using H. sapiens codon harmonized dMmuc2c4, H. sapiens optimized cytidine deaminases and H. sapiens optimized UGI, termed MmuBE_H. MmuBE_H1.A and MmuBE_H1.B contain CDA and UGI fused with a 93 aa or 16 aa linker, respectively. Using the same linker, MmuBE_H2 and MmuBE_H2YE were constructed using rAPOBEC1 and rAPOBEC1 YE instead of CDA, respectively (see Komor, Y. B. et al., (2016) Nature 533, 420-424. rAPOBEC1 YE was previously shown to have a narrower editing window (see X. Li et al., (2018) Nature Biotechnology 36: 324; Y. B. Kim et al., (2017) Nature Biotechnology 35: 371). In FIG. 14, linkers are indicated with a number, representing the amino acid length. In addition, MmuBE_E and MmuBE_H also have either an LVA degradation tag or two nuclear localization sequence (NLS) tags.

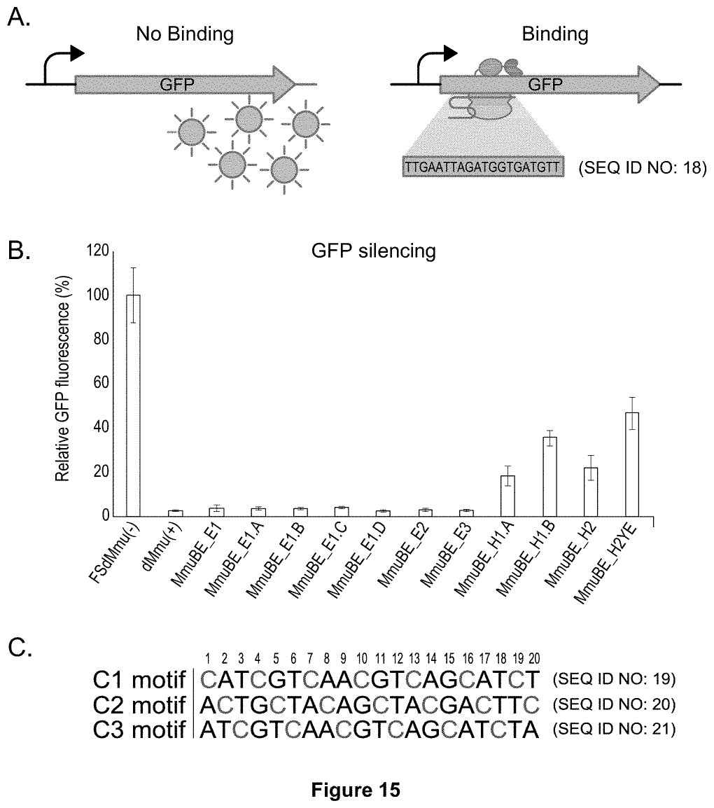

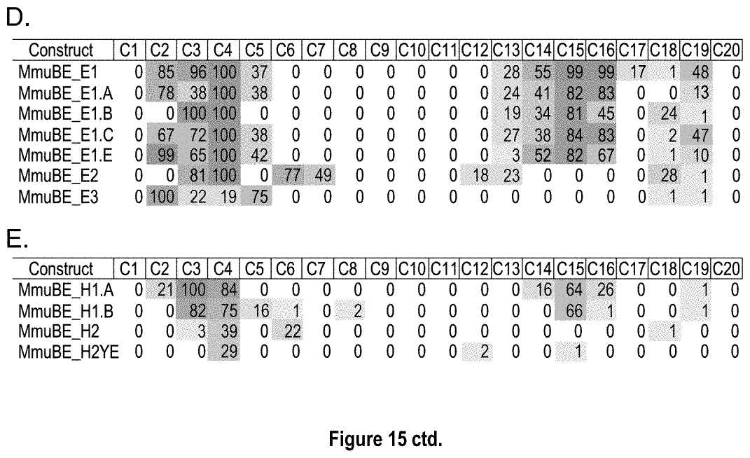

Prior to base editing, all MmuBEs were tested for binding activity of dMmu in vivo using a GFP silencing assay. MmuBEs targeted a short gfp sequence containing only D (A, G or T) nucleotides, so base editing of the target sequence cannot occur (see FIG. 15A). A frame shift E. coli dMmuC2C4 (FSdMmu) and E. coli dMmuC2c4 were included to function as negative and positive controls, respectively. GFP fluorescence was measured and normalized to FSdMmu (see FIG. 15B in which the Y-axis represents relative GFP fluorescence in % where negative control frameshift dMmu (FSdMmu) was used as 100%, and the X-axis represents the different MmuBEs tested).

From the results (see FIG. 15C), it was found that all E. coli MmuBEs (MmuBE_E) were able to bind to the target DNA, i.e. they all have <5% GFP fluorescence, so they silence GFP similarly to the positive control dMmu (E. coli harmonized). As for MmuBE_H base editors, all MmuBE_H were found to have lower silencing activity when compared to the dMmu control, with 20-50% of GFP fluorescence still being detected. Out of the MmuBE_H base editors, MmuBE_H1.A and MmuBE_H2 have the best silencing with only 18% and 22% GFP fluorescence, respectively. This is followed by MmuBE_H1.B with 35% GFP fluorescence and then MmuBE_H2YE with the least silencing of 47% of GFP fluorescence still being detected. Difference in silencing between MmuBE_E and MmuBE_H base editors can be due to expression differences affected by codon usage of E. coli. After testing the binding activity of various MmuBEs, base editing activity was tested.

### Example 10: Test for Base Editing in E coli Cells

A test for base editing was developed, by growing E. coli cells harboring pCas, pCRISPR and pTarget plasmids. pCas and pCRISPR express the base editor and the CRISPR-array, respectively, whereas pTarget plasmids contain the protospacer target. pTarget consisted of three different plasmids, named C motif plasmids. The different C motif plasmids contain a tiled C motif (CxxCxxCxxCxxCxxCxxC; SEQ ID NO: 11), starting at every first (C1 motif), second (C2 motif) or third (C3 motif) nucleotide of the protospacer (see FIG. 15C). Cells harbouring all three plasmids were grown for 48 hours and were used for a population PCR, which amplified the protospacer region on the C-motif plasmids. Amplified products were sent for Sanger Sequencing and results were analyzed by EditR (see M. G. Kluesner et al., (2018) “EditR: a method to quantify base editing from Sanger sequencing” The CRISPR Journal 1, 239-250. Base editing results obtained from all three C motif plasmids were merged and visualized using a heatmap (FIG. 15D). It was found that trimming the Mmu_BE_E1 linker from 121 aa to 24 aa (MmuBE_E1.C) had no effect on the two-region windows (FIG. 15D). However, MmuBE_E1.D, containing a 33 aa rigid linker showed slightly lower base editing in the PAM-distal region.

Unexpectedly, also MmuBE_E2 and MmuBE3, which had long flexible linkers (93 aa and 121 aa), showed reduction of the PAM-distal region. Mmu_BE2 contains a H. sapiens optimized rAPOBEC1 instead of CDA, and MmuBE3 contains a H. sapiens optimized UGI instead of the E. coli optimized UGI. Having these H. sapiens optimized genes instead can reduce overall folding of the fusion proteins thereby changing the total activity of the protein.

Next, MmuBE_H base editors were also found be active in E. coli, although they have lower base editing activity compared to MmuBE_E base editors (FIG. 15E).

MmuBE_H1.A and MmuBE_H1.13 also have two base editing regions, but with reduced overall activities. MmuBE_H1.A edits C's at position 2-4 and 14-16, whereas MmuBE_H1.B (containing a shorter linker of 16 aa) edits C's at position 3-6 and 15-16. This suggests that, in these constructs, linker reduction from 93 to 16 aa results in a slight shift of the PAM-proximal base editing region. The most precise MmuBEs in E. coli were found to be MmuBE_H2 and MmuBE_H2YE, having relatively low activity with base editing detected only in the PAM-proximal region (FIG. 2.E). MmuBE_H2 edits C's at position 3,5 and 6, whereas MmuBE_H2YE only edits at position 4 with little to no editing found at position 12 and 15. Although they have a narrow range, both MmuBE_H2 base editors have a significantly lower base editing efficiency when compared to other MmuBEs. Hence, the detected narrow base editing window appears to be a consequence of a lower editing efficiency. The reduced editing activity may have different explanations: reduced functional production of human-codon optimized dMmuC2c4 (in line with aforementioned reduction of silencing efficiency), the production and cytosine deamination activity by APOBEC, and the production and uracil glycosylase inhibition by human-codon optimized UGI.

A variety of MmuBEs were therefore created with varying in base editing windows, providing a wide selection of MmuBEs and further expanding the base editing toolbox in E. coli. In addition, several MmuBE_H base editors also showed to be promising for base editing in eukaryotic (mammalian, human) cells.

### Example 11: MmuBE Base Edits in S. cerevisiae

To check whether a MmuBE can also function in eukaryotes, a MmuBE_S was constructed and tested in Saccharomyces cerevisiae. MmuBE_S, contains a S. cerevisiae codon-optimized dMmuc2c4, a 93aa linker, and human codon-optimized variants of CDA and UGI. Apart from the S. cerevisiae optimized Mmuc2c4, MmuBE_S is similar to MmuBE_H1.A.

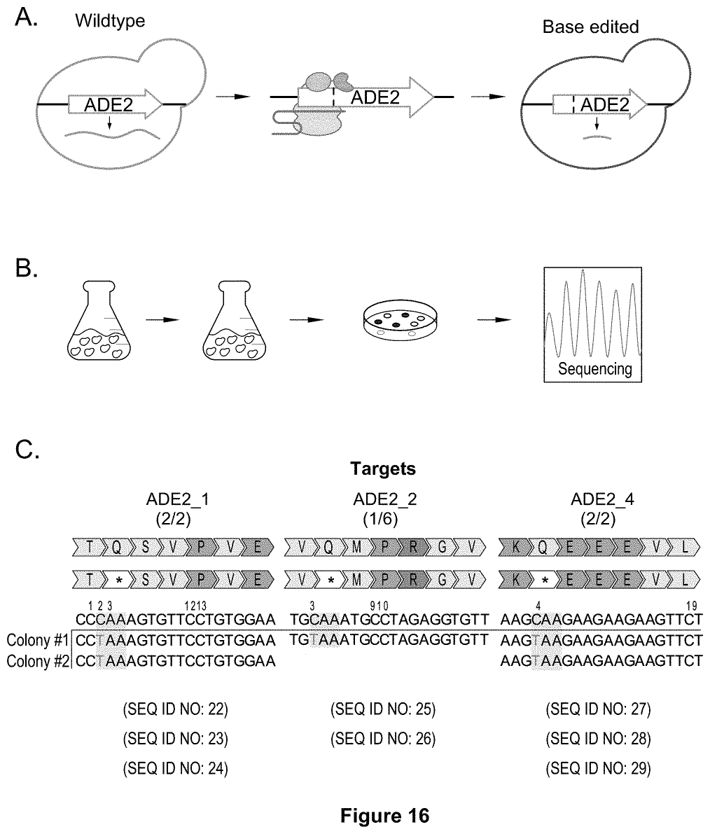

Mmu_BE_S targeted the ADE2 reporter gene in the genome of S. cerevisiae. Targeted C to T mutation in certain positions in ADE2 causes a nonsense mutation, introducing a premature stop codon. If ADE2 is knocked out, S. cerevisiae accumulates a red pigment that can be visualized as red colonies on plates, easily discriminated from the white wild type colonies (FIGS. 16A & 16B). Red colonies were selected for colony PCR and subsequent analysis of the obtained amplicons was performed by Sanger sequencing to confirm targeted base editing of the ADE2 gene (FIG. 16B). By varying the crRNA guides, MmuBE_S targeted three position in the ADE2 gene, of which C to T mutation in position 2, 3, or 4, respectively, leads to a nonsense mutation by converting a glutamine (Q) codon (CAA) to a STOP codon (TAA) (FIG. 16C). Selected colonies were sent for sequencing of the three different targets, ADE2_1, ADE2_2 and ADE2_3. From the sequencing results of the different targets, two out of two (2/2), one out of five (1/5) and two out two (2/2) were found to have the designed C to T base editing, respectively (FIG. 16C). Red colonies that did not contain targeted C to T mutations, such as the ones found in ADE2_2 and in non-targeting samples, appeared to contain either deletions or insertions, which caused a frameshift in the ADE2 gene. In addition, some red colonies were also found to have off-target base editing in the ADE2 gene, causing missense mutations, P508L and P472L (data not shown). Based on these initial analyses, targeted base editing in S. cerevisiae is possible.

### Example 12: dMmuC2c4-FokI Fusion Proteins

In the inventors experiments so far, no dsDNA cleavage activity iwas found for MmuC2c4.

To upgrade this Cas protein towards different DSB-dependent genome editing applications, dMmuC2c4 was fused to an endonuclease FokI domain. This strategy has also been previously applied to dCas9 and Cascade in order to achieve more precise, two-guided genome editing (Guilinger, J. P. (2014) “Fusion of catalytically inactive Cas9 to FokI nuclease improves the specificity of genome modification” Nature Biotechnology 32: 577 (2014); Cameron, P. et al., (2019) “Harnessing type I CRISPR-Cas systems for genome engineering in human cells” Nature Biotechnology 37: 1471-1477).

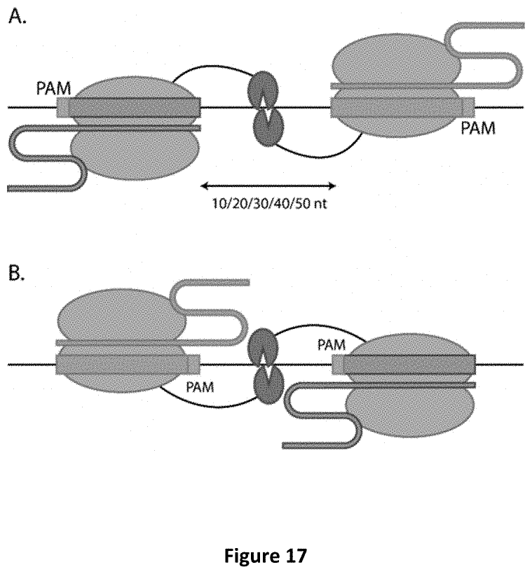

Three dMmuC2c4-FokI fusion proteins were constructed, containing a FokI domain fused at its C-terminal end. Various linker lengths were constructed consisting of 32, 98 and 121 amino acids. By guiding dMmuC2c4-FokI to target two adjacent protospacers, the FokI is brought in close proximity, allowing for their dimerization and subsequent cleavage of dsDNA. Two protospacers orientations are tested, one having the protospacers facing inwards (FIG. 17A) and the other positioning them outwards (FIG. 17B). In addition, the distance between the protospacers were also tested, varying in 10, 20, 30, 40 and 50 bp.

To test dsDNA cleavage by the dMmuC2c4-FokI proteins, a transformation assay is used. pTarget plasmids are transformed into cells expressing MmuC2c4-FokI and its CRISPR-array. If cleavage of pTarget occurs, little to no colonies is be found on the transformation plate. On the other hand, if cleavage does not occur more colonies (10-100 fold) are present on the plate. By counting the colonies of the different samples, a transformation efficiency (colony forming units/μg plasmid) is calculated and the effectiveness of dsDNA cleavage by dMmu-FokI is determined.

SEQUENCE INFORMATION
Mmu wild type Mycobacterium mucogenicum. Amino acid
[SEQ ID NO: 1]
MTTMTVHTMGVHYKWQIPEVLRQQLWLAHNLREDLVSLQLAYDDDLKAIWSSYPDVAQA
EDTMAAAEADAVALSERVKQARIEARSKKISTELTQQLRDAKKRLKDARQARRDAIAVVK
DDAAERRKARSDQLAADQKALYGQYCRDGDLYWASFNTVLDHHKTAVKRIAAQRASGK
PATLRHHRFDGSGTIAVQLQRQAGAPPRTPMVLADEAGKYRNVLHIPGWTDPDVWEQM
TRSQCRQSGRVTVRMRCGSTDGQPQWIDLPVQVHRWLPADADITGAELVVTRVAGIYR
AKLCVTARIGDTEPVTSGPTVALHLGWRSTEEGTAVATWRSDAPLDIPFGLRTVMRVDAA
GTSGIIVVPATIERRLTRTENIASSRSLALDALRDKVVGWLSDNDAPTYRDAPLEAATVKQ
WKSPQRFASLAHAWKDNGTEISDILWAWFSLDRKQWAQQENGRRKALGHRDDLYRQIA
AVISDQAGHVLVDDTSVAELSARAMERTELPTEVQQKIDRRRDHAAPGGLRASVVAAMT
RDGVPVTIVAAADFTRTHSRCGHVNPADDRYLSNPVRCDGCGAMYDQDRSFVTLMLRA
ATAPSNP
(the asterisk at the end of the sequence above signifies
a STOP codon position in the underlying nucleotide sequence.)
Mmu. wild type Mycobacterium mucogenicum. DNA
[SEQ ID NO: 2]
ATGACCACGATGACCGTGCACACCATGGGCGTGCACTACAAATGGCAGATACCTGAG
GTATTGCGCCAGCAACTGTGGCTCGCGCACAATCTGCGCGAAGACCTGGTGAGCTT
GCAGCTCGCCTACGACGACGACCTCAAAGCCATCTGGTCGTCCTACCCCGATGTGG
CCCAGGCCGAGGACACGATGGCCGCCGCAGAAGCCGACGCCGTCGCGCTGTCCGA
GCGGGTCAAGCAGGCGCGGATCGAGGCCCGGAGCAAGAAAATCAGCACCGAACTGA
CCCAACAGCTCCGCGACGCCAAGAAGCGGCTCAAGGACGCTCGCCAAGCCCGCCG
CGACGCCATCGCCGTCGTGAAAGACGATGCCGCTGAACGCCGCAAAGCGCGCAGTG
ACCAACTCGCCGCCGACCAGAAAGCGCTGTACGGGCAATACTGCCGTGACGGCGAC
CTGTACTGGGCCAGCTTCAACACGGTGCTGGACCATCACAAGACCGCAGTCAAACGT
ATTGCGGCGCAACGGGCATCGGGGAAGCCGGCGACATTGCGTCATCATCGGTTCGA
TGGCAGCGGCACCATCGCCGTGCAGCTGCAGCGCCAGGCCGGAGCGCCGCCGCGC
ACCCCCATGGTTCTCGCCGACGAGGCCGGCAAGTACCGCAACGTGTTGCACATTCC
CGGATGGACAGACCCCGATGTGTGGGAACAGATGACCCGCTCGCAATGCCGCCAAT
CCGGGCGCGTCACGGTGCGGATGCGCTGCGGCAGCACCGACGGCCAGCCACAGTG
GATCGACCTACCGGTGCAAGTGCACCGATGGCTCCCGGCCGACGCCGACATCACCG
GCGCCGAACTCGTCGTTACCCGCGTCGCCGGCATCTACCGGGCCAAGCTGTGTGTC
ACCGCCCGCATCGGAGACACAGAACCCGTCACCAGCGGACCGACCGTGGCCCTCCA
CCTGGGCTGGCGATCCACCGAGGAAGGCACCGCGGTGGCCACATGGCGCTCGGAC
GCACCCCTGGACATCCCTTTCGGCCTACGCACCGTGATGCGCGTCGATGCAGCGGG
TACGTCAGGAATCATCGTGGTGCCCGCCACCATCGAGCGCCGGCTGACACGCACAG
AAAACATCGCCTCATCCCGCTCACTGGCGCTAGATGCCTTACGCGACAAAGTCGTTG
GGTGGCTATCGGACAATGACGCACCCACCTATCGGGACGCACCGCTGGAAGCGGCA
ACAGTCAAACAGTGGAAATCGCCACAGCGATTCGCATCCCTAGCGCACGCCTGGAAA
GACAACGGCACCGAAATCTCCGACATCCTCTGGGCCTGGTTCAGCCTCGACCGAAAG
CAATGGGCCCAACAAGAAAACGGGCGCCGCAAGGCACTCGGACACCGCGACGACCT
CTACCGCCAGATCGCCGCGGTGATCAGCGACCAGGCCGGCCACGTCCTCGTCGACG
ACACCAGCGTGGCCGAACTCAGCGCCCGCGCCATGGAACGCACAGAGCTCCCAACC
GAAGTGCAACAGAAGATCGACCGGCGCCGCGATCACGCCGCCCCAGGTGGATTACG
GGCCTCCGTCGTGGCCGCCATGACCCGAGACGGCGTACCCGTAACGATCGTCGCAG
CAGCGGATTTCACACGGACACACAGCCGATGCGGCCACGTCAACCCCGCAGACGAC
CGGTACCTGTCTAACCCCGTGCGCTGCGATGGCTGCGGAGCCATGTACGACCAAGA
CCGCTCCTTTGTCACACTGATGCTGCGAGCGGCCACTGCACCATCCAATCCCTAG
Mmu. wild type Mycobacterium mucogenicum. DNA (E. coli
codon harmonized)
[SEQ ID NO: 3]
ATGACAACAATGACAGTACATACAATGGGAGTACATTATAAGTGGCAGATCCCCGAAG
TCCTTCGGCAGCAACTGTGGTTAGCACATAACCTGCGGGAAGATCTGGTATCGCTTC
AGTTAGCATATGATGATGATTTAAAGGCAATTTGGTCATCGTATCCCGACGTAGCACA
GGCAGAAGATACAATGGCAGCAGCCGAAGCAGATGCAGTTGCACTGTCGGAACGGG
TTAAACAGGCACGGATTGAAGCACGGTCGAAAAAGATTTCGACAGAACTGACACAAC
AGTTACGGGATGCAAAAAAACGGTTAAAAGATGCCCGGCAAGCACGGCGGGATGCA
ATTGCAGTTGTAAAGGATGACGCAGCCGAACGGCGGAAGGCACGGTCGGATCAATTA
GCAGCAGATCAGAAGGCACTGTATGGGCAATATTGCCGAGATGGAGATCTGTATTGG
GCATCGTTTAATACAGTACTGGATCACCATAAAACAGCCGTTAAGCGAATCGCAGCAC
AACGGGCCTCAGGGAAACCGGCAACTCTTCGACACCACCGGTTTGACGGATCGGGA
ACAATTGCAGTACAGCTGCAGCGGCAGGCAGGGGCACCGCCGCGGACACCCATGGT
CTTAGCAGATGAAGCAGGAAAATATCGGAATGTACTTCATATCCCCGGGTGGACTGAT
CCCGACGTATGGGAACAGATGACACGGTCACAATGCCGGCAATCGGGGCGGGTTAC
AGTACGGATGCGGTGCGGATCGACAGATGGACAGCCCCAGTGGATTGATCTACCGG
TACAAGTACATCGCTGGTTACCGGCAGATGCAGATATTACAGGAGCAGAATTAGTTGT
CACACGGGTTGCAGGAATTTATCGGGCAAAACTGTGTGTTACAGCACGGATTGGGGA
TACTGAACCCGTTACATCGGGGCCGACAGTAGCATTACATCTGGGATGGCGCTCGAC
AGAAGAAGGAACAGCAGTAGCAACTTGGCGGTCAGATGCCCCCCTGGATATTCCCTT
TGGACTACGGACAGTAATGCGGGTTGACGCCGCAGGGACATCGGGGATTATTGTAGT
ACCCGCAACAATTGAACGGCGGCTGACTCGGACTGAAAATATTGCATCGTCGCGGTC
GCTGGCACTAGACGCACTACGGGATAAGGTTGTCGGGTGGCTATCAGATAACGATGC
CCCCACATACCGGGATGCCCCGCTGGAAGCAGCCACTGTTAAGCAGTGGAAGTCAC
CCCAGCGCTTTGCCTCGCTAGCACATGCATGGAAGGATAATGGAACAGAAATTTCGG
ATATTTTATGGGCATGGTTTTCGTTAGATCGCAAACAATGGGCACAACAAGAAAATGG
GCGGCGGAAAGCCTTAGGGCATCGGGATGATTTATATCGGCAGATTGCAGCAGTAAT
TTCGGATCAGGCAGGACATGTTTTAGTTGATGATACATCGGTAGCAGAATTATCGGCA
CGGGCAATGGAACGGACTGAATTACCCACAGAAGTACAACAGAAAATTGATCGGCGG
CGGGACCATGCAGCACCCGGGGGGCTACGGGCATCGGTTGTAGCAGCAATGACACG
CGATGGAGTCCCCGTCACAATTGTTGCCGCCGCAGACTTTACTCGGACTCATTCGCG
CTGCGGACATGTTAATCCCGCCGATGATCGGTATCTGTCGAATCCCGTACGGTGCGA
CGGATGCGGGGCAATGTATGATCAAGATCGGTCGTTCGTTACTCTGATGCTGCGCGC
AGCAACTGCCCCCTCGAACCCCTAG
dMmu (D485A, mutation of aspartic acid at position 485 to
Alanine) Amino acid
[SEQ ID NO: 4]
MTTMTVHTMGVHYKWQIPEVLRQQLWLAHNLREDLVSLQLAYDDDLKAIWSSYPDVAQA
EDTMAAAEADAVALSERVKQARIEARSKKISTELTQQLRDAKKRLKDARQARRDAIAVVK
DDAAERRKARSDQLAADQKALYGQYCRDGDLYWASFNTVLDHHKTAVKRIAAQRASGK
PATLRHHRFDGSGTIAVQLQRQAGAPPRTPMVLADEAGKYRNVLHIPGWTDPDVWEQM
TRSQCRQSGRVTVRMRCGSTDGQPQWIDLPVQVHRWLPADADITGAELVVTRVAGIYR
AKLCVTARIGDTEPVTSGPTVALHLGWRSTEEGTAVATWRSDAPLDIPFGLRTVMRVDAA
GTSGIIVVPATIERRLTRTENIASSRSLALDALRDKVVGWLSDNDAPTYRDAPLEAATVKQ
WKSPQRFASLAHAWKDNGTEISDILWAWFSLDRKQWAQQENGRRKALGHRDDLYRQIA
AVISDQAGHVLVADTSVAELSARAMERTELPTEVQQKIDRRRDHAAPGGLRASVVAAMT
RDGVPVTIVAAADFTRTHSRCGHVNPADDRYLSNPVRCDGCGAMYDQDRSFVTLMLRA
ATAPSNP*
dMmu (D485A, mutation of aspartic acid at position 485 to
Alanine) DNA (E. coli codon harmonized)
[SEQ ID NO: 5]
ATGACAACAATGACAGTACATACAATGGGAGTACATTATAAGTGGCAGATCCCCGAAG
TCCTTCGGCAGCAACTGTGGTTAGCACATAACCTGCGGGAAGATCTGGTATCGCTTC
AGTTAGCATATGATGATGATTTAAAGGCAATTTGGTCATCGTATCCCGACGTAGCACA
GGCAGAAGATACAATGGCAGCAGCCGAAGCAGATGCAGTTGCACTGTCGGAACGGG
TTAAACAGGCACGGATTGAAGCACGGTCGAAAAAGATTTCGACAGAACTGACACAAC
AGTTACGGGATGCAAAAAAACGGTTAAAAGATGCCCGGCAAGCACGGCGGGATGCA
ATTGCAGTTGTAAAGGATGACGCAGCCGAACGGCGGAAGGCACGGTCGGATCAATTA
GCAGCAGATCAGAAGGCACTGTATGGGCAATATTGCCGAGATGGAGATCTGTATTGG
GCATCGTTTAATACAGTACTGGATCACCATAAAACAGCCGTTAAGCGAATCGCAGCAC
AACGGGCCTCAGGGAAACCGGCAACTCTTCGACACCACCGGTTTGACGGATCGGGA
ACAATTGCAGTACAGCTGCAGCGGCAGGCAGGGGCACCGCCGCGGACACCCATGGT
CTTAGCAGATGAAGCAGGAAAATATCGGAATGTACTTCATATCCCCGGGTGGACTGAT
CCCGACGTATGGGAACAGATGACACGGTCACAATGCCGGCAATCGGGGCGGGTTAC
AGTACGGATGCGGTGCGGATCGACAGATGGACAGCCCCAGTGGATTGATCTACCGG
TACAAGTACATCGCTGGTTACCGGCAGATGCAGATATTACAGGAGCAGAATTAGTTGT
CACACGGGTTGCAGGAATTTATCGGGCAAAACTGTGTGTTACAGCACGGATTGGGGA
TACTGAACCCGTTACATCGGGGCCGACAGTAGCATTACATCTGGGATGGCGCTCGAC
AGAAGAAGGAACAGCAGTAGCAACTTGGCGGTCAGATGCCCCCCTGGATATTCCCTT
TGGACTACGGACAGTAATGCGGGTTGACGCCGCAGGGACATCGGGGATTATTGTAGT
ACCCGCAACAATTGAACGGCGGCTGACTCGGACTGAAAATATTGCATCGTCGCGGTC
GCTGGCACTAGACGCACTACGGGATAAGGTTGTCGGGTGGCTATCAGATAACGATGC
CCCCACATACCGGGATGCCCCGCTGGAAGCAGCCACTGTTAAGCAGTGGAAGTCAC
CCCAGCGCTTTGCCTCGCTAGCACATGCATGGAAGGATAATGGAACAGAAATTTCGG
ATATTTTATGGGCATGGTTTTCGTTAGATCGCAAACAATGGGCACAACAAGAAAATGG
GCGGCGGAAAGCCTTAGGGCATCGGGATGATTTATATCGGCAGATTGCAGCAGTAAT
TTCGGATCAGGCAGGACATGTTTTAGTTGcaGATACATCGGTAGCAGAATTATCGGCA
CGGGCAATGGAACGGACTGAATTACCCACAGAAGTACAACAGAAAATTGATCGGCGG
CGGGACCATGCAGCACCCGGGGGGCTACGGGCATCGGTTGTAGCAGCAATGACACG
CGATGGAGTCCCCGTCACAATTGTTGCCGCCGCAGACTTTACTCGGACTCATTCGCG
CTGCGGACATGTTAATCCCGCCGATGATCGGTATCTGTCGAATCCCGTACGGTGCGA
CGGATGCGGGGCAATGTATGATCAAGATCGGTCGTTCGTTACTCTGATGCTGCGCGC
AGCAACTGCCCCCTCGAACCCCTAG
CDA Amino acid
[SEQ ID NO: 6]
MTDAEYVRIHEKLDIYTFKKQFFNNKKSVSHRCYVLFELKRRGERRACFWGYAVNKPQS
GTERGIHAEIFSIRKVEEYLRDNPGQFTINWYSSWSPCADCAEKILEWYNQELRGNGHTL
KIWACKLYYEKNARNQIGLWNLRDNGVGLNVMVSEHYQCCRKIFIQSSHNQLNENRWLE
KTLKRAEKRRSELSIMIQVKILHTTKSPAV
CDA DNA
[SEQ ID NO: 7]
ATGACCGACGCTGAGTACGTGAGAATCCATGAGAAGTTGGACATCTACACGTTTAAG
AAACAGTTTTTCAACAACAAAAAATCCGTGTCGCATAGATGCTACGTTCTCTTTGAATT
AAAACGACGGGGTGAACGTAGAGCGTGTTTTTGGGGCTATGCTGTGAATAAACCACA
GAGCGGGACAGAACGTGGCATTCACGCCGAAATCTTTAGCATTAGAAAAGTCGAAGA
ATACCTGCGCGACAACCCCGGACAATTCACGATAAATTGGTACTCATCCTGGAGTCCT
TGTGCAGATTGCGCTGAAAAGATCTTAGAATGGTATAACCAGGAGCTGCGGGGGAAC
GGCCACACTTTGAAAATCTGGGCTTGCAAACTCTATTACGAGAAAAATGCGAGGAATC
AAATTGGGCTGTGGAACCTCAGAGATAACGGGGTTGGGTTGAATGTAATGGTAAGTG
AACACTACCAATGTTGCAGGAAAATATTCATCCAATCGTCGCACAATCAATTGAATGA
GAATAGATGGCTTGAGAAGACTTTGAAGCGAGCTGAAAAACGACGGAGCGAGTTGTC
CATTATGATTCAGGTAAAAATACTCCACACCACTAAGAGTCCTGCTGTT
UGI Amino acid
[SEQ ID NO: 8]
MTNLSDIIEKETGKQLVIQESILMLPEEVEEVIGNKPESDILVHTAYDESTDENVMLLTSD
APEYKPWALVIQDSNGENKIKML
UGI DNA
[SEQ ID NO: 9]
ATGACCAACCTTTCCGACATCATAGAGAAGGAAACAGGCAAACAGTTGGTCATCCAA
GAGTCGATACTCATGCTTCCTGAAGAAGTTGAGGAGGTCATTGGGAATAAGCCGGAA
AGTGACATTCTCGTACACACTGCGTATGATGAGAGCACCGATGAGAACGTGATGCTG
CTCACGTCAGATGCCCCAGAGTACAAACCCTGGGCTCTGGTGATTCAGGACTCTAAT
GGAGAGAACAAGATCAAGATGCTA
APOBEC Amino acid
[SEQ ID NO: 10]
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHH
ADPRNRQGLRDLISSGVTIQIMTEQESGYCWRNFVNYSPSNEAHWPRYPHLWVRLYVLE
LYCIILGLPPCLNILRRKQPQLTFFTIALQSCHYQRLPPHILWATGLK
Fokl domain amino acid
[SEQ ID NO: 30]
QLVKSELEEKKSELRHKLKYVPHEYIELIEIARNSTQDRILEMKVMEFFMKVYGYRGKHLG
GSRKPDGAIYTVGSPIDYGVIVDTKAYSGGYNLPIGQADEMQRYVEENQTRNKHINPNEW
WKVYPSSVTEFKFLFVSGHFKGNYKAQLTRLNHITNCNGAVLSVEELLIGGEMIKAGTLTL
EEVRRKFNNGEINF
Fokl domain DNA sequence
[SEQ ID NO: 31]
caactagtcaaaagtgaactggaggagaagaaatctgaacttcgtcataaattgaaatatg
tgcctcatgaatatattgaattaattgaaattgccagaaattccactcaggatagaattct
tgaaatgaaggtaatggaattttttatgaaagtttatggatatagaggtaaacatttgggt
ggatcaaggaaaccggacggagcaatttatactgtcggatctcctattgattacggtgtga
tcgtggatactaaagcttatagcggaggttataatctgccaattggccaagcagatgaaat
gcaacgatatgtcgaagaaaatcaaacacgaaacaaacatatcaaccctaatgaatggtgg
aaagtctatccatcttctgtaacggaatttaagtttttatttgtgagtggtcactttaaag
gaaactacaaagctcagcttacacgattaaatcatatcactaattgtaatggagctgttct
tagtgtagaagagcttttaattggtggagaaatgattaaagccggcacattaaccttagag
gaagtcagacggaaatttaataacggcgagataaacttt

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A polynucleotide comprising a MmuC2c4 RNA endonuclease encoded by a nucleotide sequence as set forth in SEQ ID NO:2 or a sequence of at least 55% identity thereto; or SEQ ID NO: 3 or a sequence of at least 55% identity thereto.

2. An expression vector comprising a MmuC2c4 RNA endonuclease encoded by a nucleotide sequence as set forth in SEQ ID NO:2 or a sequence of at least 55% identity thereto, under the control of a suitable expression promoter.

3. An expression vector as claimed in claim 2, further comprising a nucleotide sequence comprising an expression promoter and a sequence under the control of the promoter encoding a guide RNA (gRNA) with a nucleotide sequence which recognises a target locus of interest and the PAM sequence of 5′ TTM, 5′ NTTM or 5′ CTM in dsDNA.

4. A cell comprising an expression vector of claim 2 or claim 3.

5. A cell comprising an expression vector of claim 4, further comprising a second expression vector which comprises an expression promoter and a sequence under the control of the promoter which encodes a guide RNA (gRNA) having a nucleotide sequence which recognises a target locus of interest and the PAM sequence of 5′ TTM, 5′ NTTM or 5′ CTM in dsDNA.

6. A method of repressing or interfering with the expression of a target gene sequence by an organism or cell thereof, or in a cell-free expression system, comprising exposing double stranded DNA (dsDNA) comprising the target gene sequence to an MmuC2c4 RNA endonuclease, and a guide RNA which directs the MmuC2c4 to the target gene sequence, wherein targeted binding of the MmuC2c4 endonuclease to the dsDNA results in cleavage or degradation of mRNA transcribed from the dsDNA, whilst leaving the dsDNA intact.

7. A method as claimed in claim 6, wherein the gRNA recognises a target sequence in dsDNA having a protospacer adjacent motif (PAM) sequence of 5′ TTM 5′, 5′ NTTM or 5′ CTM.

8. A method as claimed in claim 6 or claim 7, wherein the MmuC2c4 RNA endonuclease has an amino acid sequence as set forth in SEQ ID NO:1 or a sequence of at least 55% identity thereto.

9. A method as claimed in any of claims 6 to 8, wherein an organism or cell is transfected with an expression vector of claim 2, and (a) further transfected with a second expression vector which comprises an expression promoter and a sequence under the control of the promoter which encodes a guide RNA (gRNA) having a nucleotide sequence which recognises a target locus of interest and the PAM sequence of 5′ TTM 5′, 5′ NTTM or 5′ CTM in dsDNA; or (b) a gRNA having a nucleotide sequence which recognises a target locus of interest and the PAM sequence of 5′ TTM 5′, 5′ NTTM or 5′ CTM in dsDNA, is introduced directly into the organism or cell.

10. A method as claimed in any of claims 6 to 8, wherein the organism or cell is transfected with an expression vector of claim 3.

11. A method as claimed in any of claims 6 to 8, wherein a MmuC2c4 RNA endonuclease with an amino acid sequence as set forth in SEQ ID NO:1 or a sequence of at least 55% identity thereto is introduced into the cell, and wherein (i) a gRNA having a nucleotide sequence which recognises a target locus of interest and the PAM sequence of TTM 5′, 5′ NTTM or 5′ CTM in dsDNA, is also introduced into the organism or cell; or (ii) the organism or cell is transfected with an expression vector which comprises an expression promoter and a sequence under the control of the promoter which encodes a guide RNA (gRNA) having a nucleotide sequence which recognises a target locus of interest and the PAM sequence of 5′ TTM 5′, 5′ NTTM or 5′ CTM in dsDNA.

12. A method as claimed in claim 11, wherein the MmuC2c4 RNA endonuclease is introduced into the organism or cell substantially simultaneously, sequentially or separately from (i) or (ii).

13. A method as claimed in claim 11, wherein gRNA is associated with the MmuC2c4 RNA endonuclease upon introduction into the organism or cell.

14. A method as claimed in any of claims 6 to 13, wherein the organism or cell is a eukaryote.

15. An isolated RNA endonuclease comprising a catalytically inactive MmuC2c4 (dMmuC2c4) RNA endonuclease, having an amino acid sequence as set forth in SEQ ID NO:2 or a sequence of at least 55% identity thereto, wherein the endonuclease is fused to at least another protein.

16. A RNA endonuclease as claimed in claim 15, wherein the other protein is selected from an enzyme, a ligand, a marker; optionally wherein the enzyme is a cytidine deamination enzyme and/or a uracil glycosylase inhibitor (UGI); preferably wherein the RNA endonuclease is fused to both the cytidine deamination enzyme and the uracil glycosylase inhibitor (UGI).

17. A RNA endonuclease as claimed in claim 15 or claim 16, wherein the cytidine deamination enzyme and UGI are fused to the N-terminal end or C-terminal of the dMmuC2c4 endonuclease; optionally wherein the cytidine deamination enzyme is fused directly to the N-terminal end of the dMmuC2c4 endonuclease, and the UGI is fused to the cytidine deamination enzyme.

18. A RNA endonuclease as claimed in any of claims 15 to 17, which is catalytically inactive for endonuclease activity; optionally wherein the RNA endonuclease comprises a D485A substitution; preferably wherein the RNA endonuclease has an amino acid sequence as set forth in SEQ ID NO: 4 or a sequence of at least 55% identity therewith.

19. A polynucleotide comprising a nucleotide sequence encoding (i) an endonuclease inactive MmuC2c4 (dMmuC2c4) RNA endonuclease having a nucleotide sequence as set forth in SEQ ID NO:2 or a sequence of at least 55% identity therewith, (ii) a nucleotide sequence of a cytidine deamination enzyme, and (iii) a nucleotide sequence of a uracil glycosylase inhibitor (UGI), wherein the sequences of dMmuC2c4 RNA endonuclease, cytidine deamination enzyme and UGI are ordered so that the expression product of the polynucleotide is a fusion of dMmuC2c4 RNA endonuclease with cytidine deamination enzyme and UGI.

20. An expression vector comprising an expression promoter and (i) encoding a dMmuC2c4 RNA endonuclease having a nucleotide sequence as set forth in SEQ ID NO:2 or a sequence of at least 55% identity therewith, (ii) a nucleotide sequence of a cytidine deamination enzyme, and (iii) a nucleotide sequence of a uracil glycosylase inhibitor (UGI), wherein the sequences of dMmuC2c4 RNA endonuclease, cytidine deamination enzyme and UGI are ordered so that the expression product of the polynucleotide is a fusion of dMmuC2c4 RNA endonuclease with cytidine deamination enzyme and UGI.

21. An expression vector as claimed in claim 20, wherein the in frame reading order of the nucleotide sequences is (i) followed by (ii) followed by (iii).

22. An expression vector as claimed in claim 20 or claim 21, further comprising a nucleotide sequence comprising an expression promoter and a sequence under the control of the promoter encoding a guide RNA (gRNA) with a nucleotide sequence which recognises a target locus of interest and the PAM sequence of 5′ TTM, 5′ NTTM or 5′ CTM in dsDNA.

23. A RNA endonuclease as claimed in any of claims 16 to 18, a polynucleotide as claimed in claim 19, or an expression vector as claimed in any of claims 20 to 22, wherein the cytidine deamination enzyme is cytidine deaminase (CDA), apolipoprotein B mRNA editing enzyme (APOBEC) or activation-induced cytidine deaminase (AID).

24. A cell comprising an expression vector of any of claims 20 to 23.

25. A cell as claimed in claim 24, further comprising a second expression vector which comprises an expression promoter and a sequence under the control of the promoter which encodes a guide RNA (gRNA) having a nucleotide sequence which recognises a target locus of interest and the PAM sequence of 5′ TTM, 5′ NTTM or 5′ CTM in dsDNA.

26. A method of generating a C to T mutation or mutations at a target locus in a double stranded DNA (dsDNA) in an organism, cell or cell-free system, comprising exposing the dsDNA to a dMmuC2c4 RNA endonuclease of any of claims 15 to 18, and a guide RNA (gRNA) having a nucleotide sequence which recognises a target locus of interest and the PAM sequence of 5′ TTM 5′, 5′ NTTM or 5′ CTM in dsDNA.

27. A method as claimed in claim 26, wherein an organism or cell is transfected with an expression vector of any of claim 20, 21 or 23, and (a) further transfected with a second expression vector which comprises an expression promoter and a sequence under the control of the promoter which encodes a guide RNA (gRNA) having a nucleotide sequence which recognises a target locus of interest and the PAM sequence of 5′ TTM, 5′ NTTM or 5′ CTM in dsDNA; or (b) a gRNA having a nucleotide sequence which recognises a target locus of interest and the PAM sequence of 5′ TTM, 5′ NTTM or 5′ CTM in dsDNA, is introduced directly into the organism or cell.

28. A method as claimed in claim 26, wherein an organism or cell is transfected with an expression vector of claim 22 or 23.

29. A method as claimed in claim 26, wherein a MmuC2c4 RNA endonuclease with an amino acid sequence as set forth in SEQ ID NO:1 or a sequence of at least 55% identity thereto is introduced into an organism or cell, and wherein (i) a gRNA having a nucleotide sequence which recognises a target locus of interest and the PAM sequence of 5′ TTM, 5′ NTTM or 5′ CTM in dsDNA, is also introduced into the organism or cell; or (ii) the organism or cell is transfected with an expression vector which comprises an expression promoter and a sequence under the control of the promoter which encodes a guide RNA (gRNA) having a nucleotide sequence which recognises a target locus of interest and the PAM sequence of 5′ TTM, 5′ NTTM or 5′ CTM in dsDNA.

30. A method as claimed in claim 29, wherein the MmuC2c4 RNA endonuclease is introduced into the organism or cell substantially simultaneously, sequentially or separately from (i) or (ii).

31. A method as claimed in claim 30, wherein the gRNA is associated with the MmuC2c4 RNA endonuclease upon introduction of the same into the organism or cell.

32. A method as claimed in any of claims 26 to 31, wherein the organism or cell is a eukaryote.

33. A chimeric protein comprising a MmuC2c4 endonuclease having an amino acid sequence of SEQ ID NO: 1 or SEQ ID NO: 4, or a sequence of at least 55% identity thereto, and a further functional protein selected from the group consisting of a helicase, a nuclease, a helicase-nuclease, a DNA methylase, a histone methylase, an acetylase, a phosphatase, a kinase, a transcription (co-)activator, a transcription repressor, a DNA binding protein, a DNA structuring protein, a marker protein, a reporter protein, a fluorescent protein, a ligand binding protein, a signal peptide, a subcellular localisation sequence, an antibody epitope or an affinity purification tag.

34. A chimeric protein as claimed in claim 33, wherein the further functional protein is a FokI endonuclease domain; optionally wherein a linker is provided between the MmuC2c4 endonuclease and the FokI domain.

35. An expression vector comprising a polynucleotide encoding a chimeric protein as claimed in claim 33 or claim 34.


SEQUENCE LISTING (31 sequences)

SEQ ID NO: 1 - protein, 596 aa, Mycobacterium mucogenicum
MTTMTVHTMGVHYKWQIPEVLRQQLWLAHNLREDLVSLQLAYDDDLKAIWSSYPDVAQAEDTMAAAEADAVALSERVKQARIEARSKKISTELTQQLRDAKKRLKDARQARRDAIAVVKDDAAERRKARSDQLAADQKALYGQYCRDGDLYWASFNTVLDHHKTAVKRIAAQRASGKPATLRHHRFDGSGTIAVQLQRQAGAPPRTPMVLADEAGKYRNVLHIPGWTDPDVWEQMTRSQCRQSGRVTVRMRCGSTDGQPQWIDLPVQVHRWLPADADITGAELVVTRVAGIYRAKLCVTARIGDTEPVTSGPTVALHLGWRSTEEGTAVATWRSDAPLDIPFGLRTVMRVDAAGTSGIIVVPATIERRLTRTENIASSRSLALDALRDKVVGWLSDNDAPTYRDAPLEAATVKQWKSPQRFASLAHAWKDNGTEISDILWAWFSLDRKQWAQQENGRRKALGHRDDLYRQIAAVISDQAGHVLVDDTSVAELSARAMERTELPTEVQQKIDRRRDHAAPGGLRASVVAAMTRDGVPVTIVAAADFTRTHSRCGHVNPADDRYLSNPVRCDGCGAMYDQDRSFVTLMLRAATAPSNP

SEQ ID NO: 2 - DNA, 1791 bp, Mycobacterium mucogenicum
atgaccacgatgaccgtgcacaccatgggcgtgcactacaaatggcagatacctgaggtattgcgccagcaactgtggctcgcgcacaatctgcgcgaagacctggtgagcttgcagctcgcctacgacgacgacctcaaagccatctggtcgtcctaccccgatgtggcccaggccgaggacacgatggccgccgcagaagccgacgccgtcgcgctgtccgagcgggtcaagcaggcgcggatcgaggcccggagcaagaaaatcagcaccgaactgacccaacagctccgcgacgccaagaagcggctcaaggacgctcgccaagcccgccgcgacgccatcgccgtcgtgaaagacgatgccgctgaacgccgcaaagcgcgcagtgaccaactcgccgccgaccagaaagcgctgtacgggcaatactgccgtgacggcgacctgtactgggccagcttcaacacggtgctggaccatcacaagaccgcagtcaaacgtattgcggcgcaacgggcatcggggaagccggcgacattgcgtcatcatcggttcgatggcagcggcaccatcgccgtgcagctgcagcgccaggccggagcgccgccgcgcacccccatggttctcgccgacgaggccggcaagtaccgcaacgtgttgcacattcccggatggacagaccccgatgtgtgggaacagatgacccgctcgcaatgccgccaatccgggcgcgtcacggtgcggatgcgctgcggcagcaccgacggccagccacagtggatcgacctaccggtgcaagtgcaccgatggctcccggccgacgccgacatcaccggcgccgaactcgtcgttacccgcgtcgccggcatctaccgggccaagctgtgtgtcaccgcccgcatcggagacacagaacccgtcaccagcggaccgaccgtggccctccacctgggctggcgatccaccgaggaaggcaccgcggtggccacatggcgctcggacgcacccctggacatccctttcggcctacgcaccgtgatgcgcgtcgatgcagcgggtacgtcaggaatcatcgtggtgcccgccaccatcgagcgccggctgacacgcacagaaaacatcgcctcatcccgctcactggcgctagatgccttacgcgacaaagtcgttgggtggctatcggacaatgacgcacccacctatcgggacgcaccgctggaagcggcaacagtcaaacagtggaaatcgccacagcgattcgcatccctagcgcacgcctggaaagacaacggcaccgaaatctccgacatcctctgggcctggttcagcctcgaccgaaagcaatgggcccaacaagaaaacgggcgccgcaaggcactcggacaccgcgacgacctctaccgccagatcgccgcggtgatcagcgaccaggccggccacgtcctcgtcgacgacaccagcgtggccgaactcagcgcccgcgccatggaacgcacagagctcccaaccgaagtgcaacagaagatcgaccggcgccgcgatcacgccgccccaggtggattacgggcctccgtcgtggccgccatgacccgagacggcgtacccgtaacgatcgtcgcagcagcggatttcacacggacacacagccgatgcggccacgtcaaccccgcagacgaccggtacctgtctaaccccgtgcgctgcgatggctgcggagccatgtacgaccaagaccgctcctttgtcacactgatgctgcgagcggccactgcaccatccaatccctag

SEQ ID NO: 3 - DNA, 1791 bp, Artificial Sequence
  Mmu. wild type Mycobacterium mucogenicum.  DNA (E. coli codon harmonized)
atgacaacaatgacagtacatacaatgggagtacattataagtggcagatccccgaagtccttcggcagcaactgtggttagcacataacctgcgggaagatctggtatcgcttcagttagcatatgatgatgatttaaaggcaatttggtcatcgtatcccgacgtagcacaggcagaagatacaatggcagcagccgaagcagatgcagttgcactgtcggaacgggttaaacaggcacggattgaagcacggtcgaaaaagatttcgacagaactgacacaacagttacgggatgcaaaaaaacggttaaaagatgcccggcaagcacggcgggatgcaattgcagttgtaaaggatgacgcagccgaacggcggaaggcacggtcggatcaattagcagcagatcagaaggcactgtatgggcaatattgccgagatggagatctgtattgggcatcgtttaatacagtactggatcaccataaaacagccgttaagcgaatcgcagcacaacgggcctcagggaaaccggcaactcttcgacaccaccggtttgacggatcgggaacaattgcagtacagctgcagcggcaggcaggggcaccgccgcggacacccatggtcttagcagatgaagcaggaaaatatcggaatgtacttcatatccccgggtggactgatcccgacgtatgggaacagatgacacggtcacaatgccggcaatcggggcgggttacagtacggatgcggtgcggatcgacagatggacagccccagtggattgatctaccggtacaagtacatcgctggttaccggcagatgcagatattacaggagcagaattagttgtcacacgggttgcaggaatttatcgggcaaaactgtgtgttacagcacggattggggatactgaacccgttacatcggggccgacagtagcattacatctgggatggcgctcgacagaagaaggaacagcagtagcaacttggcggtcagatgcccccctggatattccctttggactacggacagtaatgcgggttgacgccgcagggacatcggggattattgtagtacccgcaacaattgaacggcggctgactcggactgaaaatattgcatcgtcgcggtcgctggcactagacgcactacgggataaggttgtcgggtggctatcagataacgatgcccccacataccgggatgccccgctggaagcagccactgttaagcagtggaagtcaccccagcgctttgcctcgctagcacatgcatggaaggataatggaacagaaatttcggatattttatgggcatggttttcgttagatcgcaaacaatgggcacaacaagaaaatgggcggcggaaagccttagggcatcgggatgatttatatcggcagattgcagcagtaatttcggatcaggcaggacatgttttagttgatgatacatcggtagcagaattatcggcacgggcaatggaacggactgaattacccacagaagtacaacagaaaattgatcggcggcgggaccatgcagcacccggggggctacgggcatcggttgtagcagcaatgacacgcgatggagtccccgtcacaattgttgccgccgcagactttactcggactcattcgcgctgcggacatgttaatcccgccgatgatcggtatctgtcgaatcccgtacggtgcgacggatgcggggcaatgtatgatcaagatcggtcgttcgttactctgatgctgcgcgcagcaactgccccctcgaacccctag

SEQ ID NO: 4 - protein, 596 aa, Artificial Sequence
  dMmu (D485A, mutation of aspartic acid at position 485 to Alanine)
MTTMTVHTMGVHYKWQIPEVLRQQLWLAHNLREDLVSLQLAYDDDLKAIWSSYPDVAQAEDTMAAAEADAVALSERVKQARIEARSKKISTELTQQLRDAKKRLKDARQARRDAIAVVKDDAAERRKARSDQLAADQKALYGQYCRDGDLYWASFNTVLDHHKTAVKRIAAQRASGKPATLRHHRFDGSGTIAVQLQRQAGAPPRTPMVLADEAGKYRNVLHIPGWTDPDVWEQMTRSQCRQSGRVTVRMRCGSTDGQPQWIDLPVQVHRWLPADADITGAELVVTRVAGIYRAKLCVTARIGDTEPVTSGPTVALHLGWRSTEEGTAVATWRSDAPLDIPFGLRTVMRVDAAGTSGIIVVPATIERRLTRTENIASSRSLALDALRDKVVGWLSDNDAPTYRDAPLEAATVKQWKSPQRFASLAHAWKDNGTEISDILWAWFSLDRKQWAQQENGRRKALGHRDDLYRQIAAVISDQAGHVLVADTSVAELSARAMERTELPTEVQQKIDRRRDHAAPGGLRASVVAAMTRDGVPVTIVAAADFTRTHSRCGHVNPADDRYLSNPVRCDGCGAMYDQDRSFVTLMLRAATAPSNP

SEQ ID NO: 5 - DNA, 1791 bp, Artificial Sequence
  dMmu (D485A, mutation of aspartic acid at position 485 to Alanine)
atgacaacaatgacagtacatacaatgggagtacattataagtggcagatccccgaagtccttcggcagcaactgtggttagcacataacctgcgggaagatctggtatcgcttcagttagcatatgatgatgatttaaaggcaatttggtcatcgtatcccgacgtagcacaggcagaagatacaatggcagcagccgaagcagatgcagttgcactgtcggaacgggttaaacaggcacggattgaagcacggtcgaaaaagatttcgacagaactgacacaacagttacgggatgcaaaaaaacggttaaaagatgcccggcaagcacggcgggatgcaattgcagttgtaaaggatgacgcagccgaacggcggaaggcacggtcggatcaattagcagcagatcagaaggcactgtatgggcaatattgccgagatggagatctgtattgggcatcgtttaatacagtactggatcaccataaaacagccgttaagcgaatcgcagcacaacgggcctcagggaaaccggcaactcttcgacaccaccggtttgacggatcgggaacaattgcagtacagctgcagcggcaggcaggggcaccgccgcggacacccatggtcttagcagatgaagcaggaaaatatcggaatgtacttcatatccccgggtggactgatcccgacgtatgggaacagatgacacggtcacaatgccggcaatcggggcgggttacagtacggatgcggtgcggatcgacagatggacagccccagtggattgatctaccggtacaagtacatcgctggttaccggcagatgcagatattacaggagcagaattagttgtcacacgggttgcaggaatttatcgggcaaaactgtgtgttacagcacggattggggatactgaacccgttacatcggggccgacagtagcattacatctgggatggcgctcgacagaagaaggaacagcagtagcaacttggcggtcagatgcccccctggatattccctttggactacggacagtaatgcgggttgacgccgcagggacatcggggattattgtagtacccgcaacaattgaacggcggctgactcggactgaaaatattgcatcgtcgcggtcgctggcactagacgcactacgggataaggttgtcgggtggctatcagataacgatgcccccacataccgggatgccccgctggaagcagccactgttaagcagtggaagtcaccccagcgctttgcctcgctagcacatgcatggaaggataatggaacagaaatttcggatattttatgggcatggttttcgttagatcgcaaacaatgggcacaacaagaaaatgggcggcggaaagccttagggcatcgggatgatttatatcggcagattgcagcagtaatttcggatcaggcaggacatgttttagttgcagatacatcggtagcagaattatcggcacgggcaatggaacggactgaattacccacagaagtacaacagaaaattgatcggcggcgggaccatgcagcacccggggggctacgggcatcggttgtagcagcaatgacacgcgatggagtccccgtcacaattgttgccgccgcagactttactcggactcattcgcgctgcggacatgttaatcccgccgatgatcggtatctgtcgaatcccgtacggtgcgacggatgcggggcaatgtatgatcaagatcggtcgttcgttactctgatgctgcgcgcagcaactgccccctcgaacccctag

SEQ ID NO: 6 - protein, 208 aa, Artificial Sequence
  CDA
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 7 - DNA, 624 bp, Artificial Sequence
  CDA
atgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtt

SEQ ID NO: 8 - protein, 84 aa, Artificial Sequence
  UGI
MTNLSDIIEKETGKQLVIQESILMLPEEVEEVIGNKPESDILVHTAYDESTDENVMLLTSDAPEYKPWALVIQDSNGENKIKML

SEQ ID NO: 9 - DNA, 252 bp, Artificial Sequence
  UGI
atgaccaacctttccgacatcatagagaaggaaacaggcaaacagttggtcatccaagagtcgatactcatgcttcctgaagaagttgaggaggtcattgggaataagccggaaagtgacattctcgtacacactgcgtatgatgagagcaccgatgagaacgtgatgctgctcacgtcagatgccccagagtacaaaccctgggctctggtgattcaggactctaatggagagaacaagatcaagatgcta

SEQ ID NO: 10 - protein, 229 aa, Artificial Sequence [sequence omitted]

SEQ ID NO: 11 - DNA, 19 bp, Artificial Sequence
  tiled C motif
  misc_feature (2)..(3): n is a, c, g, or t
  misc_feature (5)..(6): n is a, c, g, or t
  misc_feature (8)..(9): n is a, c, g, or t
  misc_feature (11)..(12): n is a, c, g, or t
  misc_feature (14)..(15): n is a, c, g, or t
  misc_feature (17)..(18): n is a, c, g, or t
cnncnncnncnncnncnnc

SEQ ID NO: 12 - DNA, 35 bp, Artificial Sequence
  Sequence in Fig. 11 B
cactactttcgcgtatggtcttcaatgctttgcga

SEQ ID NO: 13 - protein, 11 aa, Artificial Sequence
  Sequence in Fig. 11 B
TTFAYGLQCFA

SEQ ID NO: 14 - DNA, 36 bp, Artificial Sequence
  Sequence in Fig. 11 B
tcactactttcgcgtatggtctttaatgctttgcga

SEQ ID NO: 15 - protein, 7 aa, Artificial Sequence
  Sequence in Fig. 11 B
TTFAYGL

SEQ ID NO: 16 - DNA, 24 bp, Artificial Sequence
  sequence in Fig.12
tttcgcgtatggtcttcaatgctt

SEQ ID NO: 17 - DNA, 20 bp, Artificial Sequence
  sequence in Fig. 13A
ttcattaaaaattgaattga

SEQ ID NO: 18 - DNA, 20 bp, Artificial Sequence
  sequence in Fig. 15A
ttgaattagatggtgatgtt

SEQ ID NO: 19 - DNA, 20 bp, Artificial Sequence
  C1 motif
catcgtcaacgtcagcatct

SEQ ID NO: 20 - DNA, 20 bp, Artificial Sequence
  C2 motif
actgctacagctacgacttc

SEQ ID NO: 21 - DNA, 20 bp, Artificial Sequence
  C3 motif
atcgtcaacgtcagcatcta

SEQ ID NO: 22 - DNA, 20 bp, Artificial Sequence
  sequence in Fig. 16 C
cccaaagtgttcctgtggaa

SEQ ID NO: 23 - DNA, 20 bp, Artificial Sequence
  sequence in Fig. 16 C
cctaaagtgttcctgtggaa

SEQ ID NO: 24 - DNA, 20 bp, Artificial Sequence
  sequence in Fig. 16 C
cctaaagtgttcctgtggaa

SEQ ID NO: 25 - DNA, 20 bp, Artificial Sequence
  sequence in Fig. 16 C
tgcaaatgcctagaggtgtt

SEQ ID NO: 26 - DNA, 20 bp, Artificial Sequence
  sequence in Fig. 16 C
tgtaaatgcctagaggtgtt

SEQ ID NO: 27 - DNA, 20 bp, Artificial Sequence
  sequence in Fig. 16 C
aagcaagaagaagaagttct

SEQ ID NO: 28 - DNA, 20 bp, Artificial Sequence
  sequence in Fig. 16 C
aagtaagaagaagaagttct

SEQ ID NO: 29 - DNA, 20 bp, Artificial Sequence
  sequence in Fig. 16 C
aagtaagaagaagaagttct

SEQ ID NO: 30 - protein, 196 aa, Artificial Sequence
  FokI domain
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 31 - DNA, 588 bp, Artificial Sequence
  FokI domain
caactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaacttt